(19) United States
(12) Patent Application Publication
(10) Pub. No.: US 2026/0367070 A1
(43) Pub. Date: Nov. 19, 2026

(54) METHOD FOR EVALUATING QUALITY OF RETINAL GRAFT

(71) Applicant: RACTHERA Co., Ltd., Tokyo (JP)

(72) Inventors: Atsushi KUWAHARA, Kobe-shi, Hyogo (JP); Orie TERAI, Osaka-shi, Osaka (JP); Kazuki UEYAMA, Suita-shi, Osaka (JP); Aya NAKAMURA, Suita-shi, Osaka (JP)

(73) Assignee: RACTHERA Co., Ltd., Tokyo (JP)

(21) Appl. No.: 19/122,880

(22) PCT Filed: Oct. 20, 2023
(86) PCT No.: PCT/JP2023/038051

§ 371 Date: Apr. 21, 2025

(30) Foreign Application Priority Data
Oct. 21, 2022 (JP) 2022-169328

(51) Int. Cl. G01N 33/50; A61L 27/36; C12N 5/079

(52) U.S. Cl. CPC G01N 33/5023; A61L 27/3604; A61L 27/3641; C12N 5/0621; G01N 33/5082; A61L 2430/16; C12N 2506/45; C12N 2513/00; G01N 2333/4704; G01N 2333/4706

(57) ABSTRACT

The present invention aims to provide a method for evaluating the quality of a retinal graft without actually transplanting it into the retina.

The present invention relates to a method for evaluating quality of a retinal graft, comprising:
preparing at least a part of a cell population containing the retinal graft as a test sample;
culturing the test sample in the presence of a Notch signal inhibitor or retinoic acid to obtain a test cell population;
measuring a proportion of cells expressing photoreceptor cell markers, or the expression levels of (i) a photoreceptor cell maturation-related gene, (ii) a synapse formation-related gene, and (iii) a photoreceptor auxiliary gene, in the test cell population; and
comparing the measured proportion or expression level with the value measured in a control population.

### TECHNICAL FIELD

The present invention relates to a method for evaluating the quality of a retinal graft, and in particular, to a method for evaluating the quality of a retinal graft for quality control regarding differentiation ability into photoreceptor cells and/or maturation ability from photoreceptor cells, and a method for producing a retinal graft using the same.

### BACKGROUND ART

In the neural tissue of a living body, one or more types of neural cells form a layered structure. The retinal tissue, which is one type of neural tissue, is mainly composed of five types of nerve cells, namely photoreceptor cells, bipolar cells, horizontal cells, amacrine cells, and ganglion cells, and glial cells, forming a three-dimensional layered structure. Although transplantation therapy using neural tissue has been suggested to be effective as a treatment for neurological diseases, such as retinal degenerative diseases, it has been difficult to obtain tissue that maintains the layered structure and function reflecting the neural tissue of a living human body, and it has been difficult to generalize it as a treatment method. In recent years, it has become possible to produce neural tissue (e.g., retinal tissue) by differentiating it from pluripotent stem cells (Non Patent Literatures 1 to 4). Patent Literature 1 describes the use of a cell aggregate containing a neural retina and retinal pigment epithelial cells for transplantation into a living body. On the other hand, Non Patent Literature 3 states that photoreceptor (photoreceptor cell) differentiation in the culture of embryonic stem cells is a slow process delayed by Notch signaling, and can be strongly promoted by a Notch signal inhibitor, but after the treatment with the Notch signal inhibitor, the shape of the neuroretinal epithelium becomes irregular, which is considered to be due to the rapid loss of retinal progenitor cells and disruption of the tissue structure.

When using a cell population prepared for transplantation into the retina as a cellular medicinal product, it is particularly required to strictly control its quality. Known methods for evaluating the quality of retinal grafts include an image analysis method for analyzing the presence or absence of a continuous epithelial structure in the prepared cell aggregates, and a method for detecting the expression of marker genes in a sample collected from the cell aggregates (Patent Literatures 2 and 3).

### CITATION LIST

### Patent Literatures

Patent Literature 1: International Publication No. WO2019/050015
Patent Literature 2: International Publication No. WO2017/090741
Patent Literature 3: International Publication No. WO2020/184720

### Non Patent Literatures

Non Patent Literature 1: Eiraku M. et al., “Self-organized Formation of Polarized Cortical Tissues From ESCs and Its Active Manipulation by Extrinsic Signals,” Cell Stem Cell, 3(5), 519-32 (2008)
Non Patent Literature 2: Eiraku M. et al., “Self-organizing optic-cup morphogenesis in three-dimensional culture,” Nature, 472, 51-56 (2011)
Non Patent Literature 3: Nakano T. et al., “Self-formation of Optic Cups and Storable Stratified Neural Retina From Human ESCs” Cell Stem Cell, 10(6), 771-785 (2012)
Non Patent Literature 4: Kawahara A. et al., “Generation of a ciliary margin-like stem cell niche from self-organizing human retinal tissue” Nature Communications, 6, 6286 (2015)

### SUMMARY OF INVENTION

### Problems to be Solved by the Invention

Retinal grafts for transplantation into a living body may be prepared as implants at a relatively early stage of differentiation that contain a large number of retinal progenitor cells and photoreceptor precursors. In that case, it is important that differentiation into photoreceptor cells and maturation into rod photoreceptor cells and cone photoreceptor cells are induced at the transplantation site after transplantation into a living body. Conventionally, there has been no established method for evaluating the quality of retinal grafts in vitro before transplantation with respect to such differentiation ability or maturation ability after transplantation. The present invention aims to provide a method for evaluating the quality of a retinal graft without actually transplanting it into a living body.

### Means for Solution of the Problems

As a result of intensive studies to solve the above problems, the present inventors found that when at least a part of a cell population containing a retinal graft is cultured under specific conditions and the expression of differentiation or maturation markers is measured, the differentiation ability or maturation ability of the retinal graft in vivo can be evaluated, and completed the present invention. That is, the present invention provides the following method for evaluating the quality of a retinal graft and a method for producing a retinal graft.
[1] A method for evaluating quality of a retinal graft, comprising:
(1) preparing at least a part of a cell population containing the retinal graft as a test sample;
(2) culturing the test sample in the presence of a Notch signal inhibitor to obtain a test cell population;
(3) measuring a proportion of cells expressing photoreceptor cell markers including Crx and/or Recoverin in the test cell population; and
(4) comparing the proportion measured in step 3 with a proportion of cells expressing the photoreceptor cell markers measured in a control cell population.
[2] The method according to [1], further comprising:
(3′) measuring a proportion of cells expressing additional cell markers including Chx10, Pax6, and/or Sox2 in the test cell population; and
(4′) comparing the proportion measured in the step 3′ with a proportion of cells expressing the additional cell markers measured in a control cell population.
[3] The method according to [1] or [2], wherein the Notch signal inhibitor includes at least one selected from the group consisting of DAPT, crenigacestat, LY411575, dibenzazepine, RO4929097, semagacestat, and IMR-1A.
[4] The method according to any one of [1] to [3], wherein in step 2, the test sample is cultured for 2 days to 21 days.
[5] The method according to any one of [1] to [4], wherein the cell population containing the retinal graft is
   derived from pluripotent stem cells, and/or
   a cell population obtained by culturing pluripotent stem cells by an SFEB method or an SFEBq method for 50 to 90 days, and/or
   a spherical cell aggregate or a sheet-like cell aggregate.
[6] The method according to any one of [1] to [5], wherein in step 3, the proportion of the cells is measured by flow cytometry.
[7] The method according to [2], wherein in step 3′, the proportion of the cells is measured by flow cytometry.
[8] The method according to any one of [1] to [7], for identifying a retinal graft containing retinal progenitor cells having an ability to differentiate into photoreceptor cells in vivo and/or an ability to mature in vivo.
[9] The method according to any one of [1] to [8], wherein a ratio of the number of Crx-positive cells to the total number of cells in the test sample in step 1 is 5% or more and 15% or less.
[10] The method according to any one of [1] to [9], wherein the retinal graft is determined as containing retinal progenitor cells having an ability to differentiate into photoreceptor cells in vivo and/or an ability to mature in vivo in any of the following cases:
   when the proportion of cells expressing the photoreceptor cell marker in the test cell population is increased by 5% or more compared to the control cell population by difference analysis, or
   when the proportion of cells expressing the photoreceptor cell marker in the test cell population is increased by 1.2-fold or more compared to the control cell population by fold change analysis.
[11] The method according to [2], wherein the retinal graft is determined as containing retinal progenitor cells having an ability to differentiate into photoreceptor cells in vivo and/or an ability to mature in vivo in any of the following cases:
   when the proportion of cells expressing the photoreceptor cell marker in the test cell population is increased by 5% or more compared to the control cell population, and the proportion of cells expressing the additional cell marker in the test cell population is decreased by 5% or more compared to the control cell population by difference analysis, or
   when the proportion of cells expressing the photoreceptor cell marker in the test cell population is increased by 1.2-fold or more compared to the control cell population, and the proportion of cells expressing the additional cell marker in the test cell population is decreased to 0.3-fold or less compared to the control cell population by fold change analysis.
[12] A method for evaluating quality of a retinal graft, comprising:
(A) preparing at least a part of a cell population containing the retinal graft as a test sample;
(B) culturing the test sample in the presence of retinoic acid to obtain a test cell population;
(C) measuring expression levels of (i) a photoreceptor cell maturation-related gene, (ii) a synapse formation-related gene, and (iii) a photoreceptor auxiliary gene in the test cell population; and
(D) comparing the expression levels of the photoreceptor cell maturation-related gene, the synapse formation-related gene, and the photoreceptor auxiliary gene measured in step C with expression levels in the test sample before the culture of step B,
wherein the photoreceptor cell maturation-related gene includes a cone photoreceptor cell maturation-related gene including at least one selected from the group consisting of RXRG, THRB, ONECUT1, ARR3, OPN1MW (M-opsin), and OPN1SW (S-opsin), and/or a rod photoreceptor cell maturation-related gene including at least one selected from the group consisting of NRL, NR2E3, GRK1, SAG, and RHO;
the synapse formation-related gene includes at least one selected from the group consisting of SYP, CTBP2, Pikachurin, and LRIT3; and
the photoreceptor auxiliary gene includes at least one selected from the group consisting of PRPH2, GNAT1, GNAT2, and RdCVF.
[13] The method according to [12], wherein in step B, the test sample is cultured for 30 days to 150 days.
[14] The method according to [12] or [13], wherein the photoreceptor cell maturation-related gene includes RXRG, THRB, ONECUT1, ARR3, OPNIMW (M-opsin), and OPN1SW (S-opsin), and/or NRL, NR2E3, GRK1, SAG, and RHO.
[15] The method according to any one of [12] to [14], wherein the synapse formation-related gene includes SYP, CTBP2, Pikachurin, and LRIT3, and/or the photoreceptor auxiliary gene includes PRPH2, GNAT1, GNAT2, and RdCVF.
[16] The method according to any one of [12] to [15], for identifying a retinal graft containing retinal progenitor cells having the ability to mature into rod photoreceptor cells or cone photoreceptor cells in vivo.
[17] A method for producing a retinal graft, comprising:
(I) obtaining a cell population containing a retinal graft from pluripotent stem cells;
(II) evaluating quality of the retinal graft contained in the cell population obtained in step
(I) by the method according to any one of [1] to [16]; and
(III) determining whether or not the retinal graft contained in the cell population is of a quality suitable for transplantation based on the evaluation result of step (II).
[18] A method for producing a retinal graft, comprising:
(I) obtaining a cell aggregate containing a retinal graft from pluripotent stem cells;
(II) evaluating quality of the retinal graft contained in the cell aggregate obtained in step
(I) by the method according to any one of [1] to [16];
(III) determining whether or not the retinal graft contained in the cell aggregate is of a quality suitable for transplantation based on the evaluation result of step (II); and
(IV) dissecting the retinal graft determined to be of a quality suitable for transplantation in step (III) from the cell aggregate.

### Advantageous Effects of Invention

According to the present invention, by culturing at least a part of a cell population containing a retinal graft in the presence of a Notch signal inhibitor or retinoic acid and measuring the expression of differentiation or maturation markers, the differentiation ability or maturation ability of the retinal graft in vivo can be evaluated. Therefore, it becomes possible to evaluate the quality of a retinal graft without actually transplanting it into a living body.

### BRIEF DESCRIPTION OF DRAWINGS

FIG. 1 illustrates an inverted microscope image of a cell aggregate with or without a neural retina.

FIG. 2 illustrates fluorescence microscope images of immunostained cell aggregates containing neural retina.

FIG. 3A illustrates a scatter plot of Pax6-Chx10 by flow cytometry.

FIG. 3B illustrates a scatter plot of Chx10-Crx by flow cytometry, and plot diagrams of Pax6-Chx10 for Chx10-negative and Crx-positive cell subpopulations.

FIG. 4 illustrates plot diagrams of Pax6-Chx10 and Ki67-Crx by flow cytometry.

FIG. 5 illustrates plot diagrams of Sox2 or Pax6-Chx10 and Sox2-Crx by flow cytometry.

FIG. 6 illustrates a scatter plot of Pax6-Chx10 by flow cytometry.

FIG. 7 illustrates a scatter plot of Ki67-Crx by flow cytometry.

FIG. 8 illustrates results of analysis of gene expression by quantitative PCR.

FIG. 9 illustrates results of analysis of gene expression by quantitative PCR.

FIG. 10 illustrates plot diagrams of Sox2 or Pax6-Chx10 and Sox2-Crx by flow cytometry.

FIG. 11 illustrates results of analysis of gene expression by quantitative PCR.

FIG. 12 illustrates fluorescence microscope images of immunostained post-transplantation neural retinal sheets.

FIG. 13 illustrates results of analysis of gene expression by quantitative PCR.

FIG. 14 illustrates results of multivariate analysis of gene expression analyzed by quantitative PCR.

### DESCRIPTION OF EMBODIMENTS

### I. Definitions

The “retinal graft” as described herein refers to a collection of cells that can be used as a cellular medicinal product for transplantation into a living retina. The retinal graft can be prepared as a part or all of a cell population obtained by culturing cells having the ability to differentiate into photoreceptor cells. The retinal graft herein contains at least retinal progenitor cells and photoreceptor precursors, and constitutes retinal tissue such as the neural retina by inducing differentiation and maturation in vivo after transplantation into a living retina. While transplantation after differentiation and maturation to photoreceptor cells in vitro has also been considered, the quality evaluation method of the present invention is particularly useful when employing transplantation at a stage containing retinal progenitor cells and photoreceptor precursors. Since the process of differentiation and maturation does not proceed uniformly in the entire cell population and proceeds continuously in each cell, when the retinal graft contains retinal progenitor cells and photoreceptor precursors, some cells may have differentiated into photoreceptor cells.

One embodiment of the “retinal graft” may be a cell aggregate such as a spherical cell aggregate or a sheet-like cell aggregate. Cell aggregates such as spherical cell aggregates or sheet-like cell aggregates contain retinal tissue (or neural retina) at least in part.

“Spherical cell aggregate” means a cell aggregate having a three-dimensional shape close to a sphere. The three-dimensional shape close to a sphere is a shape having a three-dimensional structure, and when projected onto a two-dimensional plane, examples thereof include a spherical shape showing a circle or an ellipse, and a shape formed by a plurality of spherical shapes fused together (for example, a shape formed by overlapping two to four circles or ellipses when projected two-dimensionally). In one aspect, the core of the aggregate has a vesicular layered structure and is characterized by being observed as dark in the center and bright in the outer edge under a bright-field microscope.

“Sheet-like cell aggregate” means a two-dimensional cell aggregate having a thickness due to the formation of a layered structure by cells. For example, a two-dimensional (spread in a plane direction) cell aggregate having a thickness can be produced by cells forming a layered structure by adherent culture (two-dimensional culture) of pluripotent stem cells.

As one embodiment of the “retinal graft,” for example, by cutting out a part of a spherical cell aggregate or a sheet-like cell aggregate, a sheet-like cell aggregate prepared in a size suitable for transplantation into a human can be obtained.

“Retinal tissue” means a tissue in which one or more types of retinal cells constituting each retinal layer in a living retina exist according to a certain order, and “neural retina” is retinal tissue, and means a tissue including the inner neural retinal layer that does not include the retinal pigment epithelial layer among the retinal layers described later. In addition, the retinal tissue (or neural retina) in this specification includes immature (not fully mature) forms, for example, photoreceptor layers or neuroblastic layers containing at least retinal progenitor cells and photoreceptor precursors. In this specification, when referring to “retinal tissue” without any particular explanation, it means a concept including “neural retina.”

Retinal tissue for transplantation as a retinal graft can be prepared as a part of a cell aggregate that can be obtained by differentiating pluripotent stem cells, and specific preparation methods are described in Patent Literatures 1 and 3, and the documents cited therein. The cell aggregate containing the retinal tissue may form a specific tissue, for example, it may be a spherical cell aggregate. In addition, a plurality of retinal tissues may be present in an overlapping manner in the cell aggregate containing the retinal tissue. The retinal tissue for transplantation includes at least a photoreceptor layer or a neuroblastic layer, and the photoreceptor layer or the neuroblastic layer is formed at least on the outermost side of the cell aggregate. Retinal progenitor cells or photoreceptor precursors may also be present inside, or a photoreceptor layer or a neuroblastic layer may also be formed inside. Retinal progenitor cells and the like are present continuously in the tangential direction of the surface of the cell aggregate, that is, adhered to each other, and the presence of the retinal progenitor cells and the like continuously in the tangential direction of the surface of the cell aggregate forms a photoreceptor layer or a neuroblastic layer containing the retinal progenitor cells and the like. The tangential direction refers to the tangential direction to the surface of the cell aggregate, that is, the direction in which retinal progenitor cells and the like are aligned in the photoreceptor layer, and is the direction parallel or lateral to the retinal tissue. The tangential slope on the surface of the epithelial tissue refers to the direction in which the cells are aligned when the individual cells are aligned in a certain direction in the epithelial tissue, and is the direction parallel or lateral to the epithelial tissue (or epithelial sheet).

“Retinal cells” means cells constituting each retinal layer in a living retina or precursors thereof. Retinal cells include photoreceptor cells (rod photoreceptor cells, cone photoreceptor cells), horizontal cells, amacrine cells, interneurons, retinal ganglion cells (ganglion cells), bipolar cells (rod bipolar cells, cone bipolar cells), Müller glial cells, retinal pigment epithelial (RPE) cells, ciliary body, precursors of these (e.g., photoreceptor precursors, bipolar precursor cells, etc.), and retinal progenitor cells, but are not limited to these. Among retinal cells, cells constituting the neural retinal layer (also referred to as neural retinal cells or neural retinal system cells) specifically include photoreceptor cells (rod photoreceptor cells, cone photoreceptor cells), horizontal cells, amacrine cells, interneurons, retinal ganglion cells (ganglion cells), bipolar cells (rod bipolar cells, cone bipolar cells), Müller glial cells, and precursors of these (e.g., photoreceptor precursors, bipolar precursor cells, etc.). That is, neural retinal system cells do not include retinal pigment epithelial cells and ciliary body cells.

“Retinal progenitor cells” refers to progenitor cells that can differentiate into any immature retinal system cell—such as photoreceptor precursors, horizontal precursor cells, bipolar precursor cells, amacrine precursor cells, retinal ganglion precursor cells, Müller glial cells, and retinal pigment epithelial precursor cells—and that ultimately can give rise to any mature retinal system cell, including photoreceptor cells, rod photoreceptor cells, cone photoreceptor cells, horizontal cells, bipolar cells, amacrine cells, retinal ganglion cells, and retinal pigment epithelial cells.

“Neural retinal progenitor cells” refers to progenitor cells that can differentiate into any immature neural retinal system cell—such as photoreceptor precursors, horizontal precursor cells, bipolar precursor cells, amacrine precursor cells, retinal ganglion precursor cells, and Müller glial cells—and that ultimately can give rise to any mature neural retinal system cell, including photoreceptor cells, rod photoreceptor cells, cone photoreceptor cells, horizontal cells, bipolar cells, amacrine cells, and retinal ganglion cells. In this specification, when referring to “retinal progenitor cells” without any particular explanation, it means cells of a higher-level concept that also includes “neural retinal progenitor cells.”

“Photoreceptor precursors” refer to precursors that are determined to differentiate into photoreceptor cells.

“Photoreceptor cells” are present in the photoreceptor layer of the retina in vivo and have a role of absorbing light stimuli and converting them into electrical signals. Photoreceptor cells mature into cone photoreceptor cells that function in bright light and rod photoreceptor cells that function in dim light. Cone photoreceptor cells include S-cone photoreceptor cells that express S-opsin and detect blue light, L-cone photoreceptor cells that express L-opsin and detect red light, and M-cone photoreceptor cells that express M-opsin and detect green light. Photoreceptor cells differentiate from photoreceptor precursors and mature.

The above-mentioned cells can be identified by markers expressed by each cell. For example, markers expressed in retinal progenitor cells include Chx10 (also called Vsx2) and Pax6, and if necessary, Rx (also called Rax) and Sox2. Markers expressed in photoreceptor precursors include Crx and Blimp1. Markers expressed in bipolar cells include Chx10, PKCα, Goα, VSX1, and L7, and Chx10 is particularly strongly expressed. Markers expressed in retinal ganglion cells include TuJ1 and Brn3, markers expressed in amacrine cells include Calretinin and HPC-1, and markers expressed in horizontal cells include Calbindin. Markers expressed in photoreceptor cells and photoreceptor precursors include Crx and Recoverin. Markers expressed in rod photoreceptor cells include Rhodopsin, markers expressed in rod photoreceptor cells and rod photoreceptor precursors include NrI, and markers expressed in cone photoreceptor cells include S-opsin and LM-opsin. Markers expressed in cone photoreceptor cells, cone photoreceptor precursors, and ganglion cells include RXRG, and markers expressed in cone photoreceptor cells that appear in the early stage of differentiation or their precursors include TRβ2, OTX2, and OC2. Markers commonly expressed in horizontal cells, amacrine cells, and ganglion cells include Pax6. In one aspect of this specification, retinal progenitor cells may be cells that express Chx10 and Pax6, and may be cells that express Rx, Pax6, Sox2, and Chx10 (in this case, they may be particularly referred to as neural retinal progenitor cells). Photoreceptor precursors may be Crx-positive and Recoverin-negative cells. Crx is a marker common to photoreceptor precursors and photoreceptor cells, and in this specification, the term “Crx-positive cells” simply means a cell population that may include both photoreceptor precursors and photoreceptor cells.

“Retinal layer” means each layer constituting the retina, and specifically, the retinal pigment epithelial layer, photoreceptor layer, outer limiting membrane, outer nuclear layer, outer plexiform layer, inner nuclear layer, inner plexiform layer, ganglion cell layer, nerve fiber layer, and inner limiting membrane can be mentioned.

“Neural retinal layer” means each layer constituting the neural retina, and specifically, the photoreceptor layer, outer limiting membrane, outer nuclear layer, outer plexiform layer, inner nuclear layer, inner plexiform layer, ganglion cell layer, nerve fiber layer, and inner limiting membrane can be mentioned. The “photoreceptor layer” means a retinal layer formed on the outermost side of the neural retina and containing a large number of photoreceptor cells (rod photoreceptor cells, cone photoreceptor cells), photoreceptor precursors, and retinal progenitor cells. In view of the continuity of the formation of the layered structure, the photoreceptor layer may include a retinal progenitor cell layer in addition to the photoreceptor layer. Each layer other than the photoreceptor layer is referred to as the inner layer. Which retinal layer each cell constitutes can be confirmed by a known method, for example, the presence or absence or the degree of expression of a cell marker.

In the case of retinal tissue at a stage where the appearance rate of photoreceptor cells or photoreceptor precursors is low, a layer containing proliferating retinal progenitor cells (or neural retinal progenitor cells) is called a “neuroblastic layer,” and there are an inner neuroblastic layer and an outer neuroblastic layer. Those skilled in the art can judge by a well-known method, for example, under a bright-field microscope, by the shade of color (the outer neuroblastic layer is light and the inner neuroblastic layer is dark).

### II. Method for Evaluating the Quality of a Retinal Graft

The method for evaluating the quality of a retinal graft of the present invention comprises the steps of:
(1) preparing at least a part of a cell population containing the retinal graft as a test sample;
(2) culturing the test sample in the presence of a Notch signal inhibitor to obtain a test cell population;
(3) measuring a proportion of cells expressing photoreceptor cell markers in the test cell population; and
(4) comparing the proportion measured in step 3 with a proportion of cells expressing the photoreceptor cell markers measured in a control cell population.

Hereinafter, each step will be described in detail.

The test sample prepared in step 1 of the present invention is at least a part of a cell population containing the retinal graft, and specifically, at least a part of a cell population containing the retinal graft to be actually transplanted or a retinal graft of the same lot as the retinal graft is used. To evaluate the quality of the retinal graft in vivo, the work is complicated and time-consuming. According to the method of the present invention, since differentiation into photoreceptor cells can be accelerated and evaluated in vitro, the quality can be evaluated before transplantation or before observing the progress of the actually transplanted retinal graft, and in some cases, it is possible to take necessary measures for the recipient of the retinal graft.

The cell population containing the retinal graft can be appropriately differentiated and prepared from cells having the ability to differentiate into photoreceptor cells. The cells having the ability to differentiate into photoreceptor cells are not particularly limited, but may include, for example, pluripotent stem cells. The pluripotent stem cells are stem cells with high differentiation ability, and include, for example, embryonic stem cells (ES cells), embryonic germ cells (EG cells), induced pluripotent stem cells (iPS cells), Muse cells (multilineage differentiating stress enduring cells) obtained from mesenchymal stem cells, or GS cells produced from germ cells (e.g., testis). In one aspect, the cell population containing the retinal graft is derived from the pluripotent stem cells. For example, the cell population containing the retinal graft can be prepared by culturing the pluripotent stem cells by the SFEB method (International Publication No. WO2005/12390) or the SFEBq method (International Publication No. WO2009/148170), or an improved method thereof (for example, International Publication No. WO2015/025967, which is a production method using a BMP signaling pathway activator (BMP4)) for about 50 days to about 90 days. One embodiment of the cell population containing the retinal graft may be a cell population containing retinal progenitor cells and photoreceptor precursors, and may be a cell population at a stage where differentiation has progressed to photoreceptor precursors or photoreceptor cells at least in part. As one embodiment, the proportion of Crx-positive cells (photoreceptor precursors and photoreceptor cells) to the total number of cells in the cell population may be, for example, 5% or more and 30% or less, or 5% or more and 20% or less (about 15%+3%).

In one aspect, the cell population containing the retinal graft may be a cell aggregate such as a spherical cell aggregate or a sheet-like cell aggregate containing retinal tissue for transplantation, or a suspension for transplantation containing retinal progenitor cells or photoreceptor precursors. Preferably, the retinal graft is a cell aggregate such as a spherical cell aggregate or a sheet-like cell aggregate, or a cell aggregate obtained by cutting out a part of these. The sampling method from the cell population containing the retinal graft is not particularly limited; for example, when preparing a cell aggregate containing retinal tissue for transplantation or a part of a suspension for transplantation as a test sample for quality evaluation, a part of the cell aggregate may be cut out in the former case, and a part of the suspension may be collected in the latter case. As another example, among a plurality of retinal grafts cultured as the same lot, at least one or more retinal grafts can be used as test samples. The same lot may be, for example, a cell population cultured on the same day under the same conditions.

In step 2 of the present invention, the differentiation of retinal progenitor cells in the test sample is promoted by a Notch signal inhibitor to obtain a test cell population. Although the Notch signal inhibitor promotes differentiation into photoreceptor precursors and photoreceptor cells, the alignment of cells constituting the neuroretinal epithelium instead becomes irregular and the layered structure is lost, so that the behavior (differentiation ability) and exertion of visual function in vivo after the retinal graft is transplanted into a living body cannot be reproduced, and it was considered that it could not be used to evaluate the quality of the retinal graft in vitro. However, surprisingly, even when such a Notch signal inhibitor is used, at least a part of the cell population containing the retinal graft is used as a test sample, and by appropriately selecting the differentiation marker to be measured, and devising measures such as measuring the constituent ratio of retinal progenitor cells or photoreceptor precursors in the test cell population derived from the test sample and comparing it with the ratio measured in the control cell population, it was found that the quality of the retinal graft can be evaluated (see Examples below). Therefore, by using a Notch signal inhibitor, it becomes possible to evaluate the differentiation ability into photoreceptor cells in a short period of time, that is, at a stage before transplanting the retinal graft.

When this evaluation is performed before transplantation, the implementation period of this evaluation and the handling of the retinal tissue for transplantation or the suspension for transplantation become problems. Although it may be considered to store it as described later, it is preferable to continue culturing from the viewpoint of maintaining quality. Alternatively, when the number of culture days is fixed in the manufacturing process of the retinal graft, the test sample can be obtained by back-calculating the number of days required for this quality evaluation method. As described above, it is necessary to consider the differentiation stage of the test sample. When the culture of the retinal tissue used for transplantation is continued, the differentiation of the retinal tissue progresses, and it becomes impossible to transplant it as a retinal tissue or suspension containing a large number of retinal progenitor cells and photoreceptor precursors. Therefore, it is of great significance that evaluation in a short period of time is possible by accelerating differentiation by a method capable of evaluating differentiation ability.

The Notch signal inhibitor is not particularly limited, but may include, for example, at least one selected from the group consisting of e.g. DAPT (CAS: 208255-80-5), crenigacestat (CAS: 1421438-81-4), LY411575 (CAS: 209984-57-6), dibenzazepine (CAS: 256-96-2), RO4929097 (CAS: 847925-91-1), and semagacestat (CAS: 425386-60-3), which are γ-secretase inhibitors, and IMR-1A (CAS: 331862-41-0), which is a Notch inhibitor.

The concentration of the Notch signal inhibitor may be any concentration that can promote differentiation into photoreceptor precursors or photoreceptor cells, and may be appropriately adjusted according to the test cell population and culture conditions. As one embodiment, in the case of DAPT, it may be about 1 to about 100 μM. The concentration of other Notch signal inhibitors may be a concentration that exhibits a Notch signal inhibitory effect comparable to that of the above DAPT concentration. The Notch signal inhibitory effect can be measured by a conventional method such as a binding activity evaluation to a target protein (γ-secretase, Notch, etc.).

As the medium used in step 2 of the present invention, any medium commonly used in the art can be used without particular limitation except that it contains the Notch signal inhibitor, but for example, the medium may contain a medium suitable for culturing nerve cells. For example, a medium that can be used for culturing animal cells, such as BME medium, BGJb medium, CMRL 1066 medium, Glasgow MEM (GMEM) medium, Improved MEM Zinc Option medium, IMDM medium, Medium 199 medium, MEM medium, Eagle MEM medium, αMEM medium, DMEM medium, F-12 medium, DMEM/F12 medium, IMDM/F12 medium, Ham's medium, RPMI 1640 medium, Fischer's medium, Leibovitz's L-15 medium, or a mixed medium thereof, can be mentioned. A medium supplemented with serum or N2 medium, which is a supplementary medium, may also be used. As one embodiment, DMEM/F12 medium supplemented with 10% fetal bovine serum, 1% N2 supplement, and 100 μM taurine can be mentioned. The medium used in step 2 may contain other components as long as they do not inhibit the differentiation promoting effect of the Notch signal inhibitor on photoreceptor cells or photoreceptor precursors.

The Notch signal inhibitor may be used together with one or more substances selected from FGF signal activators, FGF signal inhibitors, EGF signal activators, EGF signal inhibitors, WNT signal activators, WNT signal inhibitors, Shh signal activators, and Shh signal inhibitors in the culture of nerve cells and stem cells. Since the purpose of the culture in step 2 of the present invention is different from that of conventional cultures using these, the culture can be performed in the absence of one or more substances selected from FGF signal activators, FGF signal inhibitors, EGF signal activators, EGF signal inhibitors, WNT signal activators, WNT signal inhibitors, Shh signal activators, and Shh signal inhibitors.

The culture period in step 2 of the present invention is not particularly limited as long as it can induce the differentiation of retinal progenitor cells and photoreceptor precursors, but for example, the test sample may be cultured for about 2 days to about 21 days or about 7 days to about 14 days.

In step 3 of the present invention, the proportion of cells expressing photoreceptor cell markers including Crx and/or Recoverin in the test cell population is measured. As a method for measuring the proportion of such cells, any method commonly used in the art can be used without particular limitation, but for example, the proportion of the cells may be measured by an immunological method such as flow cytometry. Flow cytometry is particularly preferable for quantitative evaluation. This point also applies to the measurement of other cell markers.

In step 4 of the present invention, the proportion of cells measured in step 3 is compared with a proportion of cells expressing the photoreceptor cell markers measured in a control cell population. If the former proportion is higher than the latter, it is determined that the retinal graft from which the test sample is derived has a good differentiation ability into photoreceptor cells and/or is suitable for transplantation. The control cell population may be appropriately prepared; for example, the test sample prepared in step 1 may be used as a control cell population without culturing in step 2 or after storage (e.g., after cryopreservation), or the test sample may be cultured under the same conditions as in step 2 except for culturing in the absence of the Notch signal inhibitor to obtain a control cell population.

In one aspect, the proportion of cells expressing cell markers that are not expressed by photoreceptor cells but are expressed by retinal progenitor cells in the test cell population may be further measured; specifically, (step 3′) the proportion of cells expressing additional cell markers including Chx10, Pax6, and/or Sox2 is further measured, and (step 4′) the measured proportion of cells is compared with a proportion of cells expressing the additional cell markers measured in the control cell population. If the former proportion is lower than the latter, it is determined that the retinal graft from which the test sample is derived has a good differentiation ability. As described in the Examples below, it was shown that the photoreceptor cell marker and the additional cell marker are expressed in the cells shown in Table 1 below, respectively; therefore, by measuring a proportion of cells expressing these markers together, it becomes possible to evaluate the quality of the retinal graft from which the test sample is derived in more detail.

TABLE 1
Cell markers and cells expressing them

| Cell type | Crx | Recoverin | Chx10 | Pax6 | Sox2 |
|---|---|---|---|---|---|
| Retinal progenitor cells | − | − | + | + | + |
| Photoreceptor precursors | ++ | − | − | − | − |
| Photoreceptor cells | ++ | + | − | − | − |
| Bipolar precursor cells | + | n.d. | ++ | − | − |
| Ganglion cells and amacrine | − | − | − | ++ | − |
| cells | | | | | |

n.d.: not determined

In one aspect, the proportion of cells expressing cell markers expressed by rod photoreceptor precursors or cone photoreceptor precursors in the test cell population may be further measured; specifically, the proportion of cells expressing differentiation markers such as rod photoreceptor precursor markers (such as NRL) and/or cone photoreceptor precursor markers (such as RXRG) may be measured, and the measured cell proportion may be compared with the proportion of cells expressing the maturation cell markers measured in the control cell population.

Difference analysis and/or fold change can be performed on the control cell population and the test cell population. Difference analysis means the difference between the expression ratio of a specific marker in the test sample and the expression ratio of the same marker in the control cell population. Fold change analysis means the ratio of the expression ratio of a specific marker in the test sample to the expression ratio of the same marker in the control cell population. For example, when the proportion of cells expressing the photoreceptor cell marker in the test cell population is increased by about 5% or more compared to the control cell population by difference analysis, or when the proportion of cells expressing the photoreceptor cell marker in the test cell population is increased by about 1.2-fold or more (for example, 1.5-fold or more, or 2-fold or more) compared to the control cell population by fold change analysis, the retinal graft may be determined as containing retinal progenitor cells having an ability to differentiate into photoreceptor cells in vivo and/or an ability to mature in vivo. In one embodiment, when the proportion of cells expressing the photoreceptor cell marker in the test cell population is increased by about 5% or more (e.g., 10% or more) compared to the control cell population, and the proportion of cells expressing the additional cell marker in the test cell population is decreased by about 5% or more compared to the control cell population by difference analysis, or when the proportion of cells expressing the photoreceptor cell marker in the test cell population is increased by about 1.2-fold or more (increased by about 20% or more) compared to the control cell population, and the proportion of cells expressing the additional cell marker in the test cell population is decreased to about 0.3-fold or less (decreased by about 70% or more) compared to the control cell population by fold change analysis, the cell implant may be determined as containing retinal progenitor cells having an ability to differentiate into photoreceptor cells in vivo and/or an ability to mature in vivo.

In one aspect, after step 4 of the present invention, a pharmaceutical composition or a therapeutic agent can be prepared from a retinal graft determined as having good differentiation ability into photoreceptor cells and/or as being suitable for transplantation, or a retinal graft of the same lot as the retinal graft. During the above-mentioned quality evaluation period, the retinal graft from which the test sample under evaluation is derived or the retinal graft of the same lot may be temporarily stored (refrigerated or frozen) as it is, or may be stored after being prepared as a pharmaceutical composition or a therapeutic agent. Alternatively, the culture of the retinal graft may be continued during the above-mentioned quality evaluation period, and by doing so, the quality of the retinal graft can be maintained well. By transplanting a retinal graft determined as being suitable for transplantation by this method, it is possible to suppress variations in quality due to manufacturing and to use only retinal grafts exhibiting good therapeutic efficacy for transplantation.

The pharmaceutical composition or therapeutic agent can be used for the treatment of diseases based on disorders of neural retinal system cells or neural retina, or damage to the neural retina. Examples of diseases based on disorders of neural retinal system cells or neural retina include ophthalmic diseases such as retinal degenerative diseases, macular degeneration, age-related macular degeneration, retinitis pigmentosa, glaucoma, corneal diseases, retinal detachment, central serous chorioretinopathy, cone dystrophy, and cone-rod dystrophy. Examples of the damaged state of the neural retina include a state in which photoreceptor cells are degenerated and dead.

The evaluation results obtained by the method of the present invention correlate with the maturation into rod photoreceptor cells and cone photoreceptor cells, and the expression of photoreceptor cell-related marker genes described later (Test Example: 6), and also correlate with photoreceptor cell maturation after transplantation (Test Example: 7). Therefore, by confirming differentiation into photoreceptor cells by the method of the present invention, it is possible to evaluate the subsequent maturation ability into rod photoreceptor cells and cone photoreceptor cells, and other photoreceptor cell maturation abilities (formation of photoreceptor cell synapses, formation of outer segments, and expression of genes related to photoreceptor cell signal transduction including outer segments).

In another aspect, the method for evaluating the quality of a retinal graft of the present invention comprises the steps of:
(A) preparing at least a part of a cell population containing the retinal graft as a test sample;
(B) culturing the test sample in the presence of retinoic acid to obtain a test cell population;
(C) measuring expression levels of genes related to differentiation and maturation in the test cell population; and
(D) comparing the expression levels of the gene measured in step C with expression levels in the test sample before the culture of step B.

Hereinafter, each step will be described in detail.

Step A of the present invention is as described for step 1 of the present invention. According to the method of the present invention, since the maturation into rod photoreceptor cells and cone photoreceptor cells can be evaluated in vitro, the quality can be evaluated before observing the progress of the actually transplanted retinal graft, and in some cases, it is possible to take necessary measures for the recipient of the retinal graft.

In step B of the present invention, retinoic acid promotes the differentiation and maturation of photoreceptor precursors and photoreceptor cells in the test sample to obtain a test cell population. As the medium used in this step B, any medium commonly used in the art can be used without particular limitation except that it contains the retinoic acid; for example, the medium may contain medium suitable for nerve cell culture. Specifically, the media that can be used for the culture of animal cells described above can be mentioned. A medium supplemented with serum or N2 medium, which is a supplementary medium, may also be used. For example, DMEM/F12 GlutaMAX medium supplemented with 10% fetal bovine serum, 1% N2 supplement, 100 μM taurine, and 0.5 μM retinoic acid can be mentioned. The medium used in step B may contain other components as long as they do not inhibit the differentiation promoting effect of retinoic acid on photoreceptor cells.

The culture period in step B of the present invention is not particularly limited as long as it can induce the differentiation and maturation of retinal progenitor cells and photoreceptor precursors, but for example, the test sample may be cultured for about 30 days to about 150 days, about 40 days to about 120 days, or about 50 days to about 90 days. It was found that the expression of the genes described later can be evaluated particularly stably and quantitatively after the elapse of the culture days under the conditions.

In step C of the present invention, the expression levels of (i) a photoreceptor cell maturation-related gene, (ii) a synapse formation-related gene, and (iii) a photoreceptor auxiliary gene are measured in the test cell population. As a method for measuring the expression levels of these genes, any method commonly used in the art can be used without particular limitation, but for example, the expression level of the gene may be measured by a PCR method, a semi-quantitative PCR method, or a quantitative PCR method such as a real-time PCR method.

The photoreceptor cell maturation-related gene includes a cone photoreceptor cell maturation-related gene and/or a rod photoreceptor cell maturation-related gene. The former cone photoreceptor cell maturation-related gene includes at least one selected from the group consisting of RXRG, THRB, ONECUT1, ARR3, OPNIMW (M-opsin), and OPN1SW (S-opsin). The latter rod photoreceptor cell maturation-related gene includes at least one selected from the group consisting of NRL, NR2E3, GRK1, SAG, and RHO. The synapse formation-related gene includes at least one selected from the group consisting of SYP, CTBP2, Pikachurin, and LRIT3. The photoreceptor auxiliary gene includes at least one selected from the group consisting of PRPH2, GNAT1, GNAT2, and RdCVF.

In step D of the present invention, the expression level of each gene measured in step C is compared with the expression level in the test sample before the culture of step B. If the former expression level is higher than the latter, it is determined that the retinal graft from which the test sample is derived has good differentiation and maturation ability into rod photoreceptor cells and/or cone photoreceptor cells. In one aspect, when the expression level of each gene measured in step C is compared with the expression level in the test sample before the culture of step B by fold change analysis, it may be determined that the retinal graft has a good differentiation and maturation ability into rod photoreceptor cells and/or cone photoreceptor cells when the gene expression level is increased by about 3-fold or more or about 4-fold or more.

As shown in the Test Examples, a retinal graft determined as having differentiation and maturation ability into photoreceptor cells by each method of the present invention exhibits differentiation and maturation ability in vivo after transplantation. Therefore, according to each method of the present invention, the quality can be evaluated easily, quickly, and with high reproducibility, compared to a method in which the retinal graft is actually transplanted into a living body and differentiated and matured into cells that exhibit therapeutic efficacy before evaluating the quality. In addition, according to each method of the present invention, quantitative evaluation of quality is also possible. In particular, the great advantage of the present invention is that it is possible to evaluate the differentiation and maturation ability in vivo after transplantation by selecting a good retinal graft before transplantation, or to predict the differentiation and maturation state of the retinal graft after transplantation into a human. Since the environment in vitro and in vivo is significantly different, it was unclear whether the differentiation and maturation state of the retinal graft after transplantation could be evaluated based on the evaluation in vitro, but in the present invention, it has been verified that the state after transplantation can be evaluated.

In addition, each method of the present invention may further comprise any step that can be performed for cell culture, preparation of a retinal graft, or quality evaluation, as long as it does not impair the object of the present invention, and may be performed in combination with other methods for evaluating the quality of a retinal graft.

As one embodiment of the combination, it can be combined with a fundus examination and an OCT examination after transplantation. In each method of the present invention, after evaluating the differentiation and maturation potential of the retinal graft, the engraftment of the retinal graft is confirmed by a fundus examination and an OCT examination after transplantation (e.g., 7 to 30 days after transplantation), so that the effect of insufficient engraftment can also be considered, and the effect in the patient can be evaluated more accurately.

### III. Method for Producing a Retinal Graft

The method for evaluating the quality of a retinal graft described above in connection with the present invention can be used as one step in producing a retinal graft. In other words, the present invention also relates to a method for producing a retinal graft, comprising:
(I) obtaining a cell population containing a retinal graft from pluripotent stem cells;
(II) evaluating quality of the retinal graft contained in the cell population obtained in step
(I) by the method for evaluating the quality of a retinal graft described above in connection with the present invention; and
(III) determining whether or not the retinal graft contained in the cell population is of a quality suitable for transplantation based on the evaluation result of step (II).

In one aspect, the step (I) may comprise a step of preparing the cell population comprising the retinal graft.

In one aspect of the production method of the present invention, the method may further comprise a step of dissecting a retinal graft determined to be of a quality suitable for transplantation in the step (III) from a cell aggregate. For example, when the cell population is a cell aggregate such as a spherical cell aggregate or a sheet-like cell aggregate, a part of these can be dissected. That is, there may be a case where a step of dissecting a retinal graft from a cell aggregate containing a retinal graft determined as having good differentiation ability into photoreceptor cells and/or as being suitable for transplantation is included. Those skilled in the art can appropriately select the position and size to be dissected. Patent Literature 3 and the like can be referred to for the dissection position, size, and method of the retinal graft. As one embodiment, a region near the center of the continuous epithelial tissue in the spherical cell aggregate or the sheet-like cell aggregate can be dissected as a retinal graft.

Hereinafter, the present invention will be specifically described with reference to Examples, but the scope of the present invention is not limited to these Examples.

### EXAMPLES

### Test Example 1: Production of Cell Aggregates Containing Neural Retina

Human iPS cells (DSP-SQ strain, established at Sumitomo Pharma Co., Ltd.) were established using a commercially available Sendai virus vector (four factors of Oct3/4, Sox2, KLF4, and c-Myc, CytoTune kit manufactured by ID Pharma Co., Ltd.) based on the method described in the public protocol of Thermo Fisher Scientific Inc. (iPS 2.0 Sendai Reprogramming Kit, Publication Number MAN0009378, Revision 1.0) and the public protocol of Kyoto University (Establishment and maintenance culture of human iPS cells in feeder-free conditions, CiRA_Ff-iPSC_protocol_JP_v140310, http://www.cira.kyoto-u.ac.jp/j/research/protocol.html) using StemFit medium (AK03; manufactured by Ajinomoto Co., Inc.) and Laminin511-E8 (manufactured by Nippi, Inc.).

The human iPS cells (DSP-SQ strain) were cultured under feeder-free conditions according to the method described in Scientific Reports, 9, 18936 (2019). StemFit medium (AK03N, manufactured by Ajinomoto Co., Inc.) was used as the feeder-free medium, and Laminin511-E8 (manufactured by Nippi, Inc.) was used as the feeder-free scaffold.

As a specific maintenance culture operation, first, the human iPS cells (DSP-SQ strain) that became subconfluent were washed with PBS and then dispersed into single cells using TrypLE Select (manufactured by Life Technologies). Then, the human iPS cells dispersed into the single cells were seeded on a plastic culture dish coated with Laminin511-E8 and cultured under feeder-free conditions in StemFit medium in the presence of Y27632 (ROCK inhibitor, 10 μM). When a 6-well plate (manufactured by Iwaki Co., Ltd., for cell culture, culture area 9.4 cm²) was used as the plastic culture dish, the number of human iPS cells dispersed into the single cells was set to 1.0×10⁴. One day after seeding, the medium was replaced with StemFit medium not containing Y27632. Thereafter, the medium was replaced with StemFit medium not containing Y27632 once every one to two days. Then, the cells were cultured until 5 days after seeding.

As a differentiation induction operation, human iPS cells (DSP-SQ strain) were cultured under feeder-free conditions using StemFit medium until two days before subconfluence (to the extent that 30% of the culture area was covered with cells). The human iPS cells two days before the subconfluence were cultured under feeder-free conditions for two days in the presence of SAG (300 nM) (Precondition treatment).

The human iPS cells subjected to the Precondition treatment were treated with a cell dispersion solution using TrypLE Select (manufactured by Life Technologies) and further dispersed into single cells by pipetting operation. Then, the human iPS cells dispersed into the single cells were suspended in 100 μl of serum-free medium at 1×10⁴ cells per well of a non-cell-adhesive 96-well culture plate (PrimeSurface 96V bottom plate, manufactured by Sumitomo Bakelite Co., Ltd.) and cultured in suspension at 37 °C and 5% CO2. As the serum-free medium (gfCDM+KSR) at that time, a serum-free medium prepared by adding 10% KSR, 450 μM 1-monothioglycerol, and 1×Chemically defined lipid concentrate to a 1:1 mixture of F-12 medium and IMDM medium was used. At the start of suspension culture (day 0 after the start of suspension culture), Y27632 (final concentration: 20 μM) and SAG (final concentration: 10 nM) were added to the serum-free medium. On day 2 after the start of suspension culture, 50 μl of the new serum-free medium was added so that the final concentration of exogenous human recombinant BMP4 was 1.5 nM (55 ng/ml) in a medium containing human recombinant BMP4 (manufactured by R&D Systems) without Y27632 and SAG.

Four days later (6 days after the start of suspension culture), the medium was replaced with the serum-free medium not containing Y27632, SAG, and human recombinant BMP4. As the operation of medium replacement, 60 μl of the medium in the incubator was discarded, and 90 μl of the new serum-free medium was added to make the total medium volume 180 μl. Thereafter, half of the medium was replaced with the serum-free medium not containing Y27632, SAG, and human recombinant BMP4 once every 2 to 4 days. As the half medium replacement operation, 90 μl, which is half the volume of the medium in the incubator, was discarded, and 90 μl of the new serum-free medium was added to make the total medium volume 180 μl.

The cell aggregates obtained on day 13 after the start of suspension culture were cultured in a serum-free medium (medium prepared by adding 1% N2 supplement to DMEM/F12 medium) containing CHIR99021 (3 μM) and SU5402 (5 μM) for 3 days, that is, until day 16 after the start of suspension culture.

The obtained cell aggregates on day 16 after the start of suspension culture were cultured in a serum medium shown in [1], [2], and [3] below under 5% CO₂ conditions until day 75 after the start of suspension culture.
[1] From day 16 to day 40 after the start of suspension culture: a medium prepared by adding 10% fetal bovine serum, 1% N2 supplement, and 100 μM taurine to DMEM/F12 medium (hereinafter referred to as medium A).
[2] From day 40 to day 60 after the start of suspension culture: a medium prepared by mixing medium A and a medium prepared by adding 10% fetal bovine serum, 2% B27 supplement, 2 mM glutamine, 60 nM T3, and 100 AM taurine to Neurobasal medium (hereinafter referred to as medium B) at a ratio of 1:3.
[3] After day 60 after the start of suspension culture: medium B.

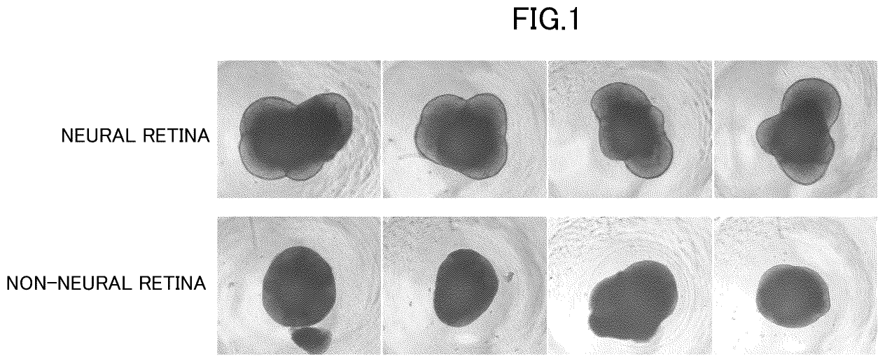

The cell aggregates on day 76 after the start of suspension culture were observed for bright-field images (phase-contrast images) with an inverted microscope (manufactured by Olympus Corporation, IX-83). The obtained image is shown in FIG. 1.

FIG. 1 was observed by focusing on the morphology of each cell and the characteristics of the adhesion state between cells. In the cell aggregate, a site where the cells had a continuous epithelial structure and the outer neuroblastic layer (including the photoreceptor layer and the neural retinal progenitor cell layer) and the inner neuroblastic layer were distinguished into two layers was determined as the neural retina. In addition, a tissue in which a continuous epithelial structure was not observed, or a site where a continuous epithelial structure was observed but the outer neuroblastic layer and the inner neuroblastic layer could not be distinguished and appeared as one layer, was determined as a non-neural retina.

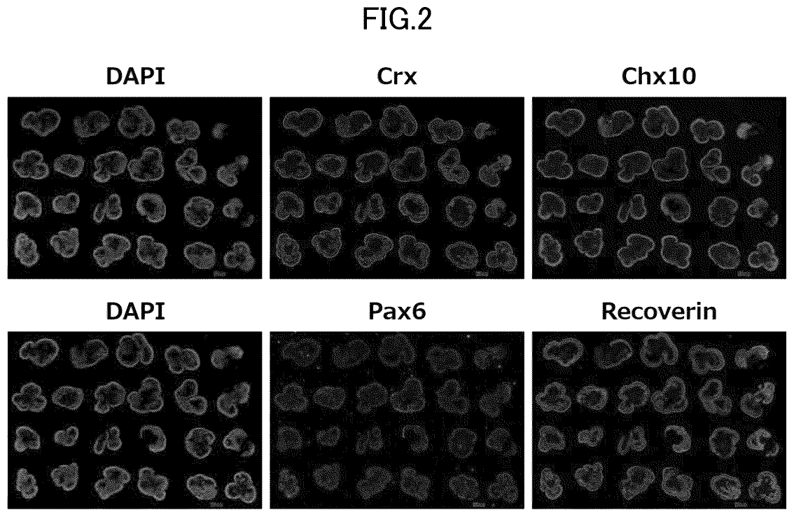

Cell aggregates determined as neural retina were fixed with 4% paraformaldehyde on day 80 after the start of suspension culture, and frozen sections were prepared. The frozen sections were immunostained for Chx10 (anti-Chx10 antibody, Exalpha, sheep), which is one of the neural retinal markers, and Crx (anti-Crx antibody, Takara, rabbit), which is one of the markers for photoreceptor precursors and photoreceptor cells. Another frozen section was immunostained for Rx (anti-Rx antibody, Takara, guinea pig), which is one of the neural retinal markers, and Recoverin (anti-Recoverin antibody, Proteintech, rabbit), which is one of the photoreceptor cell markers. Cell nuclei were stained with DAPI. The stained sections were observed using a fluorescence microscope (manufactured by Keyence Corporation), and immunostained images were acquired. The acquired image is shown in FIG. 2.

From the DAPI stained image of FIG. 2, it was found that a neural tissue in which cells were densely packed was formed on the surface of the cell aggregate, and the neural tissue formed a continuous epithelial structure. As a result of analyzing the Crx/Chx10 stained image of FIG. 2, it was found that a Crx-positive layer (photoreceptor layer) was formed on the surface of the cell aggregate, and a Chx10-positive layer (neural retinal progenitor cell layer) was formed inside the Crx-positive layer in this neural tissue (FIG. 2). Furthermore, as a result of analyzing the Pax6/Recoverin stained image of FIG. 2, it was found that a Recoverin-positive layer (photoreceptor layer) was formed, and a Pax6-positive layer (ganglion cell and amacrine cell layer) was formed inside in this neural tissue. From the positional relationship between the Crx/Chx10 stained image and the Pax6/Recoverin stained image, it was found that the Pax6-positive layer was formed further inside the Chx10-positive layer. From these results, it was found that in this neural tissue, a photoreceptor layer containing Crx-positive cells and Recoverin-positive cells was formed on the surface, a neural retinal progenitor cell layer containing Chx10-positive cells was formed inside the photoreceptor layer, and a ganglion cell and amacrine cell layer was also formed inside the neural retinal progenitor cells. That is, it was found that a neural retina containing a photoreceptor layer and a neural retinal progenitor cell layer can be produced from human iPS cells by this production method, and this neural retina has a continuous epithelial structure.

### Test Example 2: Quantitative Method for Various Neural Retinal Cells

Neural retina was differentiated from human iPS cells by the method described in Test Example 1, and cell aggregates on day 70 after the start of suspension culture were obtained.

The cells were washed with HBSS, and enzymatically treated with a nerve cell dispersion solution (manufactured by WAKO, containing papain) at 37 °C for about 20 minutes to obtain a single cell suspension. The number of cells in the single cell suspension was counted with a cell counter. The single cell suspension was fixed using a fixative (manufactured by BD Biosciences, CytoFix, containing Paraformaldehyde) to obtain a sample for flow cytometry (hereinafter referred to as FCM). The FCM sample was blocked and permeabilized using a Perm/Wash solution containing serum (manufactured by BD Biosciences).

The permeabilized cell suspension was immunostained by reacting with an antigen-antibody at 4 °C for 60 minutes in the dark using the antibody set shown in [1] below. An Isotype control antibody was used as a negative control for immunostaining.
[1] Anti-Chx10 antibody (manufactured by Santa Cruz Biotechnology, Alexa 488-conjugated) and anti-Pax6 antibody (manufactured by BD Biosciences, PE-conjugated), which are neural retinal markers, and anti-Crx antibody (manufactured by Santa Cruz Biotechnology, Alexa 647-conjugated), which is a photoreceptor precursor and photoreceptor cell marker.

The immunostained cell suspension was measured using an analyzer (manufactured by BD Biosciences). The obtained results were analyzed using a scatter plot of forward scattered light and side scattered light in flow cytometry, and the analysis target was set to a single cell fraction.

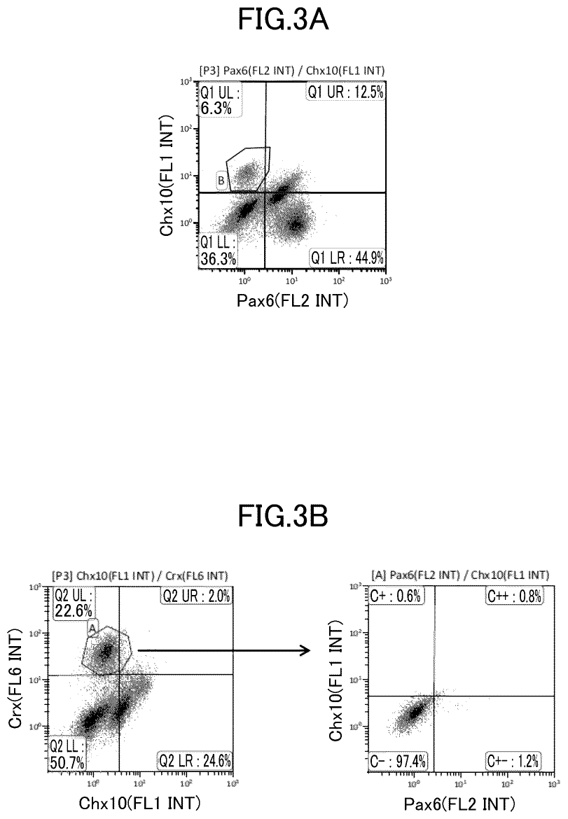

The measurement results are shown in FIGS. 3A and 3B. The analysis was performed by dividing into the following cell populations [1-1], [1-2], [1-3], and [1-4].
[1-1] Neural retinal progenitor cells: Pax6-positive and Chx10-positive cell population
[1-2] Bipolar precursor cells: Pax6-negative and Chx10-positive cell population
[1-3] Ganglion cells and amacrine cells: Pax6-positive and Chx10-negative cell population
[1-4] Photoreceptor precursors and photoreceptor cells: Crx-positive cell population

From the scatter plot of Pax6-Chx10 (FIG. 3A), it was found that the cell populations of [1-1], [1-2], and [1-3] can be detected. In addition, it was found from the scatter plot of Chx10-Crx (FIG. 3B, left figure) that the cell population of [1-4] can be detected. In addition, it was found that the population of photoreceptor precursors and photoreceptor cells specified in [1-4] was Pax6-negative and Chx10-negative when analyzed by drawing it into a scatter plot of Pax6-Chx10 (FIG. 3B, right figure). Therefore, it was found that the population of photoreceptor precursors and photoreceptor cells of [1-4] was mutually exclusive with the cell populations of [1-1], [1-2], and [1-3]. Therefore, it was demonstrated that four types of cells constituting the neural retina, namely neural retinal progenitor cells, bipolar precursor cells, ganglion cells and amacrine cells, and photoreceptor precursors and photoreceptor cells, can be specifically detected.

### Test Example 3: Evaluation Method for Photoreceptor Cell Differentiation Ability

Cell aggregates containing neural retina were selected from the cell aggregates obtained by the method described in Test Example 1. Then, while observing with a stereomicroscope, the neural retina was dissected from the cell aggregate under a stereomicroscope using fine-tipped tweezers and scissors to prepare 11 neural retinal sheet samples. Of these neural retinal sheet samples, a total of 11 samples, 7 neural retinal sheet samples were divided into a first sample, and 4 samples were divided into a second sample. Then, the tissue samples were stored at 17 °C for 3 days in a solution prepared by adding Gentacin (antibiotic) to a commercially available storage solution.

The first sample (7 samples) was cultured using medium B at 37 °C under 5% CO2 conditions for 7 to 11 days (cell population 1). The second sample (4 samples) was cultured in a medium prepared by adding DAPT (Notch signal inhibitor) to medium B at a final concentration of 10 μM (hereinafter referred to as medium C) under 5% CO₂ conditions for the same number of days as population 1, which is 7 to 11 days (cell population 2).

Cell population 1 and cell population 2 after culture were subjected to dispersion, fixation, and staining by the method described in Test Example 2. However, the antibody sets shown in [2] and [3] below were used for staining. An Isotype control antibody was used as a negative control for immunostaining.
[2] Anti-Pax6 antibody (manufactured by BD Biosciences, Alexa 488-conjugated), anti-Sox2 antibody (manufactured by BD Biosciences, PE-conjugated), and anti-Chx10 antibody (manufactured by Santa Cruz Biotechnology, Alexa 647-conjugated), which are neural retinal markers.
[3] Anti-Ki67 antibody (manufactured by BD Biosciences, Alexa 488-conjugated), which is a proliferating cell marker, anti-Sox2 antibody (manufactured by BD Biosciences, PE-conjugated), which is a glial marker, and anti-Crx antibody (manufactured by Santa Cruz Biotechnology, Alexa 647-conjugated), which is a photoreceptor precursor and photoreceptor cell marker.

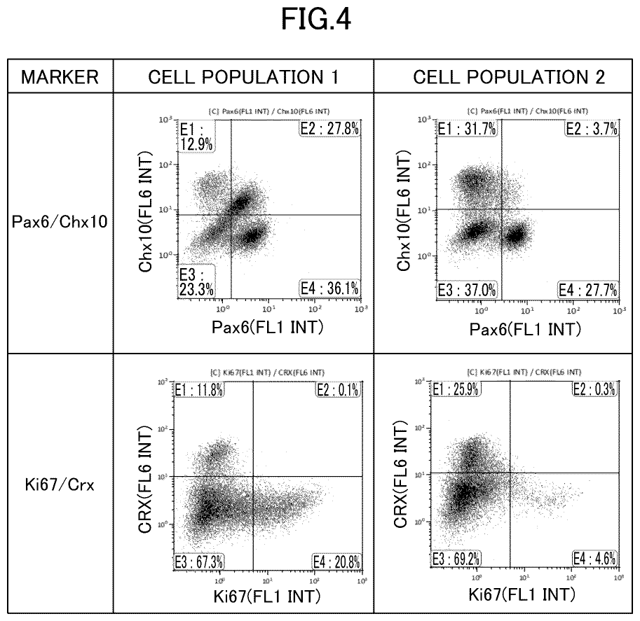

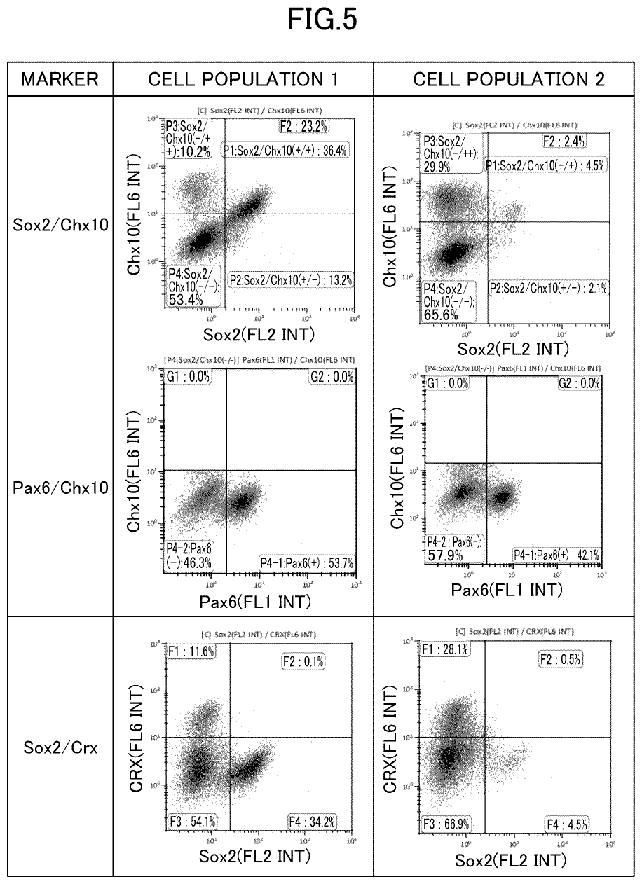

Then, measurement was performed using an analyzer (manufactured by BD Biosciences). The measurement results are shown in FIGS. 4 and 5. From the measurement results of forward scattered light and side scattered light, single cells were analyzed, and analyzed by analysis method [2-1] or [2-2] and analysis method [3] in flow cytometry. The proportion of cells was calculated by the difference from the Isotype control for each cell type. The results analyzed by analysis method [2-1] and [3] are shown in Table 2, and the results analyzed by analysis method [2-2] and [3] are shown in Table 3.
[2-1] For the results obtained by staining with antibody set [2], Pax6-positive and Chx10~ positive cells were analyzed as neural retinal progenitor cells, Pax6-negative and Chx10-positive cells were analyzed as bipolar precursor cells, and Pax6-positive and Chx10-negative cells were analyzed as ganglion cells.
[2-2] For the results obtained by staining with antibody set [2], Sox2-positive cells were analyzed as neural retinal progenitor cells, Sox2-negative and Chx10-positive cells were analyzed as bipolar precursor cells, and Sox2-negative, Chx10-negative, and Pax6-positive cells were analyzed as ganglion cells.
[3] For the results obtained by staining with antibody set [3], Crx-positive cells were analyzed as photoreceptor precursors and photoreceptor cells.

TABLE 2
Proportion of each cell type

| | Cell | Cell |
|---|---|---|
| Cell type | population 1 | population 2 |
| Neural retinal progenitor cells | 27.8% | 3.7% |
| Pax6+/Chx10+ | | |
| Bipolar precursor cells Pax6−/Chx10+ | 12.9% | 31.7% |
| Ganglion cells and amacrine cells | 24.5% | 24.8% |
| Pax6+/Chx10− | | |
| Photoreceptor precursors and photoreceptor | 11.8% | 25.9% |
| cells Crx+ | | |
| Total | 77% | 86.1% |

TABLE 3
Proportion of each cell type

| | Cell | Cell |
|---|---|---|
| Cell type | population 1 | population 2 |
| Neural retinal progenitor cells | 34.3% | 3.9% |
| Sox2+/Chx10+ | | |
| Bipolar precursor cells Sox2−/Chx10+ | 10.2% | 29.9% |
| Ganglion cells and amacrine cells | 23.8% | 24.5% |
| Pax6+/Sox2−/Chx10+ | | |
| Photoreceptor precursors and photoreceptor | 11.6% | 28.1% |
| cells Crx+ | | |
| Total | 79.9% | 86.4% |

In the results of FIG. 4, the proportion of three types of cells, neural retinal progenitor cells, bipolar precursor cells, and ganglion cells, can be quantified by two markers, Pax6 and Chx10, and the proportion of photoreceptor precursors and photoreceptor cells can be quantified from the analysis of the Crx marker, as in Test Example 2.

In FIG. 4, cells can be distinguished into four cell populations by the combination of positive and negative of two markers, Pax6 and Chx10, but since the neural retinal progenitor cell population is weakly positive for Pax6 and Chx10, there is a variation in fluorescence intensity for each experiment, and it was not easy to distinguish the cell population negative for both markers with a specific threshold. Therefore, it was found that the neural retinal progenitor cell population was better distinguished by using the Sox2 marker (FIG. 5, upper panel). Therefore, it was found that neural retinal progenitor cells, bipolar precursor cells, and ganglion cells can be precisely distinguished by performing analysis method [2-2].

Furthermore, as shown in FIG. 5, lower panel, it was found that Crx-positive cells are Sox2-negative. Therefore, it was found that photoreceptor precursors and photoreceptor cells can be quantified mutually exclusively with neural retinal progenitor cells, bipolar precursor cells, and ganglion cells even in analysis method [3].

As shown in Table 3, since the total of four types of retinal cells is 79.9% in cell population 1, about 80% of the produced neural retinal sheet is composed of neural retinal progenitor cells, bipolar precursor cells, ganglion cells, and photoreceptor precursors and photoreceptor cells.

In Table 3, focusing on neural retinal progenitor cells, the proportion was 34.3% in cell population 1 (control group, DAPT non-added group), whereas it was significantly decreased to 3.9% in cell population 2 (DAPT added group). Focusing on photoreceptor precursors and photoreceptor cells, the proportion was 11.6% in cell population 1, whereas it was significantly increased to 28.1% in cell population 2. These changes in proportion indicate that DAPT (Notch signal inhibitor) addition decreases neural retinal progenitor cells and increases photoreceptor precursors and photoreceptor cells, that is, it shows a differentiation promoting effect on the photoreceptor cell differentiation lineage from neural retinal progenitor cells. Therefore, it was demonstrated that in this test system, the photoreceptor cell differentiation ability in response to DAPT treatment can be quantitatively evaluated for neural retina containing neural retinal progenitor cells. In addition, it was found that in cell population 2 compared to cell population 1, bipolar precursor cells increased, and ganglion cells and amacrine cells were at the same level with no significant change. Therefore, it was demonstrated that by using this test system, it is possible to evaluate the differentiation propensity from neural retinal progenitor cells to various retinal cells such as photoreceptor cells, bipolar cells, and ganglion cells and amacrine cells.

### Test Example 4: Evaluation of Photoreceptor Cell Differentiation Ability

Whether or not it is possible to appropriately detect the presence or absence of photoreceptor cell differentiation ability was verified by comparing neural retinal and non-neural retinal samples.

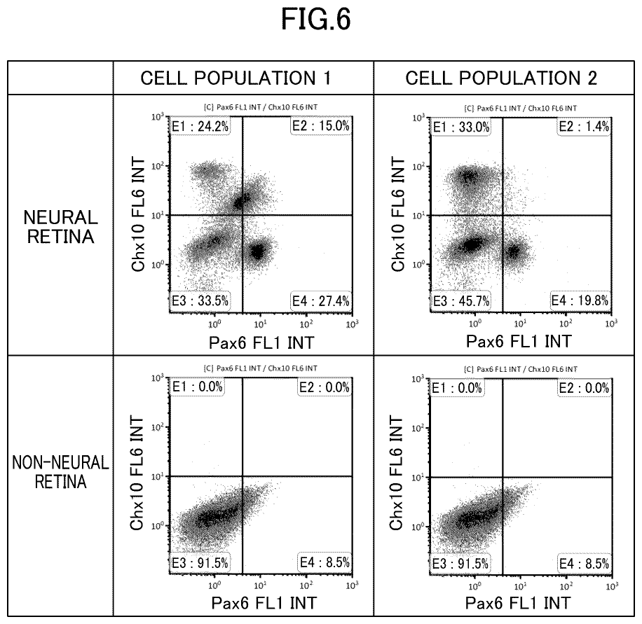

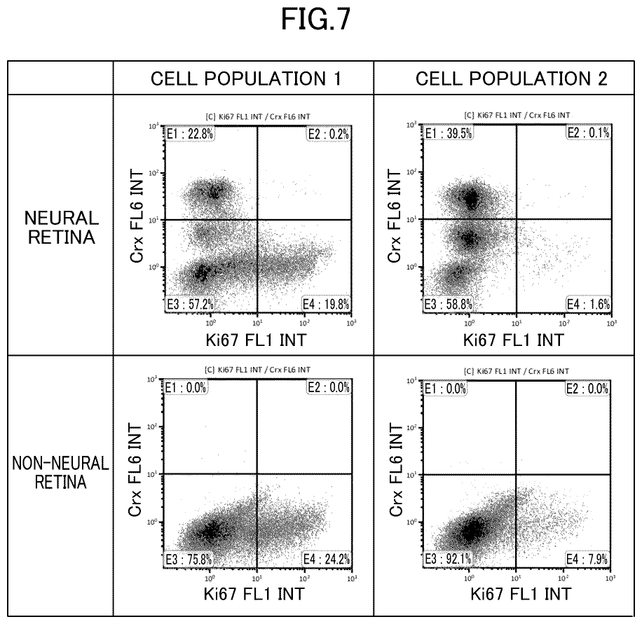

Cell aggregates were obtained by the method described in Test Example 1, and neural retina and non-neural retina were selected. A total of 10 neural retinal sheet samples and 12 non-neural retinal sheet samples were obtained from the neural retina and non-neural retina, respectively, by the method described in Test Example 3. Six neural retinal sheet samples were used as the first sample, and four neural retinal sheet samples were used as the second sample; similarly, six non-neural retinal sheet samples were used as the first sample, and six non-neural retinal sheet samples were used as the second sample. Then, the tissue samples were stored for 3 days in a solution prepared by adding Gentacin (antibiotic) to a commercially available storage solution. Cells were dispersed, fixed, stained, measured, and analyzed by the method described in Test Example 3. The measurement results of the neural retinal sheet and the non-neural retinal sheet are shown in FIGS. 6 and 7, respectively. The analysis was performed by analysis method [2-1] and analysis method [3]. The calculated results are shown in Tables 4 and 5.

TABLE 4
Results of neural retinal sheet

| | Cell population | | | Fold |
|---|---|---|---|---|
| Cell type | 1 | 2 | Difference | change |
| Neural retinal progenitor cells | 15.0% | 1.4% | −13.6% | 0.09 |
| Pax6+/Chx10+ | | | | |
| Bipolar precursor cells | 24.2% | 33.0% | +8.8% | 1.36 |
| Pax6−/Chx10+ | | | | |
| Ganglion cells and amacrine | 25.2% | 18.4% | −6.8% | 0.73 |
| cells Pax6+/Chx10− | | | | |
| Photoreceptor precursors and | 22.7% | 39.3% | +16.6% | 1.73 |
| photoreceptor cells Crx+ | | | | |
| Total | 87.1% | 92.1% | | |

TABLE 5
Results of non-neural retinal sheet

| | Cell population | | | Fold |
|---|---|---|---|---|
| Cell type | 1 | 2 | Difference | change |
| Neural retinal progenitor cells | 0.0% | 0.0% | ±0% | N.A. |
| Pax6+/Chx10+ | | | | |
| Bipolar precursor cells | 0.0% | 0.0% | ±0% | N.A. |
| Pax6−/Chx10+ | | | | |
| Ganglion cells and amacrine cells | 4.9% | 3.4% | −1.5% | 0.69 |
| Pax6+/Chx10− | | | | |
| Photoreceptor precursors and | 0.0% | 0.0% | ±0% | N.A. |
| photoreceptor cells Crx+ | | | | |
| Total | 4.9% | 3.4% | | |

As shown in Table 4, in cell population 1 (control group) containing the neural retinal sheet, the proportion of neural retinal progenitor cells as well as the proportion of photoreceptor precursors and photoreceptor cells were higher than the respective proportions in cell population 1 containing the non-neural retinal sheet shown in Table 5; in particular, even under the condition of no Notch signal inhibitor (under the condition where differentiation is not induced), the proportion of neural retinal progenitor cells was 10% or more, and the proportion of photoreceptor precursors and photoreceptor cells was 5% or more, so it is considered that the neural retinal sheet used in this test was an appropriate neural retina to be used as a test sample. In this neural retinal sheet, neural retinal progenitor cells decreased and photoreceptor precursors and photoreceptor cells increased by DAPT (Notch signal inhibitor) treatment, and thus promotion of photoreceptor cell differentiation ability was confirmed. More specifically, according to difference analysis, in cell population 2 (DAPT added group), neural retinal progenitor cells decreased by 5% or more and photoreceptor precursors and photoreceptor cells increased by 5% or more compared to cell population 1 (control group). In addition, according to fold change, in cell population 2 (DAPT added group), neural retinal progenitor cells decreased to 0.3-fold or less (decreased by 70% or more) and photoreceptor precursors and photoreceptor cells increased by 1.2-fold or more (increased by 20% or more) compared to cell population 1 (control group). On the other hand, as shown in Table 5, photoreceptor precursors and photoreceptor cells did not increase by DAPT (Notch signal inhibitor) treatment in the non-neural retinal sheet, and photoreceptor cell differentiation ability was not confirmed. From this result, it was demonstrated that the presence or absence of photoreceptor cell differentiation ability can be distinguished by this evaluation method.

Furthermore, similar tests were repeated, and when difference analysis was performed, if neural retinal progenitor cells decreased by 5% or more and photoreceptor precursors and photoreceptor cells increased by 5% or more in cell population 2 (DAPT added group) compared to cell population 1 (control group), it was considered that the neural retinal sheet (retinal graft) from which the test sample was derived had photoreceptor cell differentiation ability. In addition, when fold change was performed, if neural retinal progenitor cells decreased to 0.3-fold or less (decreased by 70% or more) and photoreceptor precursors and photoreceptor cells increased by 1.2-fold or more (increased by 20% or more) in cell population 2 (DAPT added group) compared to cell population 1 (control group), it was considered that the neural retinal sheet (retinal graft) from which the test sample was derived had photoreceptor cell differentiation ability. According to these criteria, the non-neural retinal sheet was not found to have photoreceptor cell differentiation ability by any of the analysis methods, and it was considered that it could not be used as a retinal graft. From these results, it was demonstrated that the decrease in neural retinal progenitor cells and the increase in photoreceptor precursors indeed mean photoreceptor cell differentiation ability in both the aforementioned difference analysis and fold change analysis.

### Test Example 5: Evaluation Method for Photoreceptor Cell Maturation Ability

As a new evaluation method that can distinguish whether a neural retinal sheet has photoreceptor cell maturation ability, it was decided to culture the neural retinal sheet for a long period of time and examine the gene expression of mature photoreceptor cell markers.

Cell aggregates were obtained by the method described in Test Example 1, and neural retina and non-neural retina were selected. Among the non-neural retinas, tissues expressing Chx10 (neural retinal progenitor cell marker), Crx, and Recoverin (photoreceptor cell markers) were selected as morphologically abnormal neural retina. Three neural retinal sheet samples, three morphologically abnormal neural retinal sheet samples, and two non-neural retinal sheet samples were obtained from the neural retina, morphologically abnormal neural retina, and non-neural retina, respectively, by the method described in Test Example 3. After dissecting each sheet, the adjacent position of the sheet dissected from the cell aggregate was dissected under a stereomicroscope using fine-tipped tweezers and scissors to form a ring. Three neural retinal ring samples, three morphologically abnormal neural retinal ring samples, and two morphologically abnormal ring samples were prepared.

Neural retinal sheets, morphologically abnormal neural retinal sheets, and non-neural retinal sheets were cultured at 37 °C under 5% CO₂ conditions until approximately day 180 after the start of suspension culture using medium B described in Test Example 1 or medium D below to obtain mature neural retinal sheets, mature morphologically abnormal neural retinal sheets, and mature non-neural retinal sheets, respectively. [4] Medium D: Medium prepared by adding 10% fetal bovine serum, 1% N2 supplement, 100 μM taurine, and 0.5 μM retinoic acid to DMEM/F12 GlutaMAX medium.

Total RNA was extracted from mature neural retinal sheets, mature morphologically abnormal neural retinal sheets, mature non-neural retinal sheets, neural retinal rings, morphologically abnormal neural retinal rings, and morphologically abnormal rings, and reverse-transcribed into cDNA using a reverse transcriptase and a primer (Reverse Transcription Master Mix Kit, manufactured by Fluidigm Corporation). The cDNA was subjected to a multiplex-PCR reaction (Pre-Run) using a PCR device (ProFlex PCR system, manufactured by Applied Biosystems) with all probes used for verification. Then, the Pre-Run reaction solution was injected into a multi-well with a flow path (96.96 Dynamic Array IFC, manufactured by Fluidigm Corporation) using an IFC controller HX (manufactured by Standard BioTools), and the expression level of mature photoreceptor cell marker genes was measured by real-time PCR using a multi-sample real-time PCR system (Biomark HD, manufactured by Standard BioTools). The PCR probes used for the measurement are shown in Table 6.

TABLE 6
Measured genes and their PCR probes

| Category | Gene | Probe ID | GenBank ID |
|---|---|---|---|
| Internal standard | GAPDH | Hs02758991_g1 | NM_001256799.2; NM_001289745.1 |
| | | | NM_001289746.1; NM_002046.5 |
| | ActinB | Hs01060665_g1 | NM_001101.3 |
| | HPRT1 | Hs02800695_m1 | NM_000194.2 |
| Neural retinal | Chx10 | Hs01584047_m1 | NM_182894.2 |
| progenitor cells | | | |
| Photoreceptor cells | CRX | Hs00230899_m1 | NM_000554.4 |
| | RCVRN | Hs00610056_m1 | NM_002903.2 |
| Mature | RXRG | Hs00199455_m1 | NM_001256570.1; NM_001256571.1 |
| photoreceptor cell | | | NM_006917.4 |
| genes | THRB | Hs00230861_m1 | NM_000461.4; NM_001128176.2 |
| (Cone | | | NM_001128177.1; NM_001252634.1 |
| photoreceptor cell | | | XM_005265421.4; XM_005265423.4 |
| genes) | | | XM_005265424.3; XM_006713317.3 |
| | | | XM_006713318.3; XM_011534046.2 |
| | | | XM_011534047.2; XM_011534048.2 |
| | | | XM_011534049.2; XM_011534050.2 |
| | | | XM_011534051.2; XM_011534052.2 |
| | | | XM_011534053.2; XM_017007108.1 |
| | | | XM_017007109.1; XM_017007110.1 |
| | | | XM_017007111.1; XM_017007112.1 |
| | | | XM_017007113.1; XM_017007114.1 |
| | | | XM_017007115.1 |
| | ONECUT1 | Hs00413554_m1 | NM_004498.2 |
| | ARR3 | Hs01020134_m1 | NM_004312.2; XM_017029518.1 |
| | OPN1MW | Hs04194752_g1 | NM_000513.2 |
| | OPN1SW | Hs00181790_m1 | NM_001708.2 |
| Mature | NRL | Hs00172997_m1 | NM_006177.3; XM_005267708.4 |
| photoreceptor cell | | | XM_005267709.3; XM_005267710.3 |
| genes | | | XM_011536801.2; XM_011536802.1 |
| (Rod photoreceptor | | | XM_011536804.2; XM_011536805.2 |
| cell genes) | NR2E3 | Hs00183915_m1 | NM_014249.3; NM_016346.3 |
| | GRK1 | Hs00943051_m1 | NM_002929.2; XM_017020684.1 |
| | SAG (s-arrestin) | Hs01027187_m1 | NM_000541.4; XM_011511593.2 |
| | | | XM_011511594.1; XM_011511596.1 |
| | | | XM_017004641.1; XM_017004642.1 |
| | | | XM_017004643.1; |
| | RHO | Hs00892431_m1 | NM_000539.3 |
| Synapse formation- | SYP | Hs00300531_m1 | NM_003179.2 |
| related genes | (Synaptophysin) | | |
| | CTBP2 | Hs00949547_g1 | NM_001083914.2; NM_001290214.2 |
| | | | NM_001290215.2; NM_001321012.1 |
| | | | NM_001321013.1; NM_001321014.1 |
| | | | NM_001329.3; NM_022802.2 |
| | | | XM_005269561.2; XM_005269564.2 |
| | | | XM_005269567 2; XM_005269568.4 |
| | | | XM_005269569.2; XM_005269571.2 |
| | | | XM_005269572 3; XM_006717642.2 |
| | | | XM_011539349.2; XM_011539351.1 |
| | | | XM_011539353.1; XM_011539354.1 |
| | | | XM_011539355.1; XM_011539357.1 |
| | | | XM_011539358.1; XM_017015756.1 |
| | | | XM_017015757.1 |
| | EGFLAM | Hs00794831_m1 | NM_001205301.1; NM_152403.3 |
| | (Pikachurin) | | NM_182798.2; NM_182801.2 |
| | LRIT3 | Hs01651050_m1 | NM_198506.4; XM_017008167.1 |
| Photoreceptor | PRPH2 | Hs00165616_m1 | NM_000322.4 |
| signal transduction | TXNL6 (RdCVF, | Hs00370782_m1 | NM_138454.1 |
| genes | NXNL1) | | |
| | GNAT1 | Hs00181100_m1 | NM_000172.3; NM_144499.2 |
| | GNAT2 | Hs00292542_m1 | NM_005272.3; XM_011541264.2 |

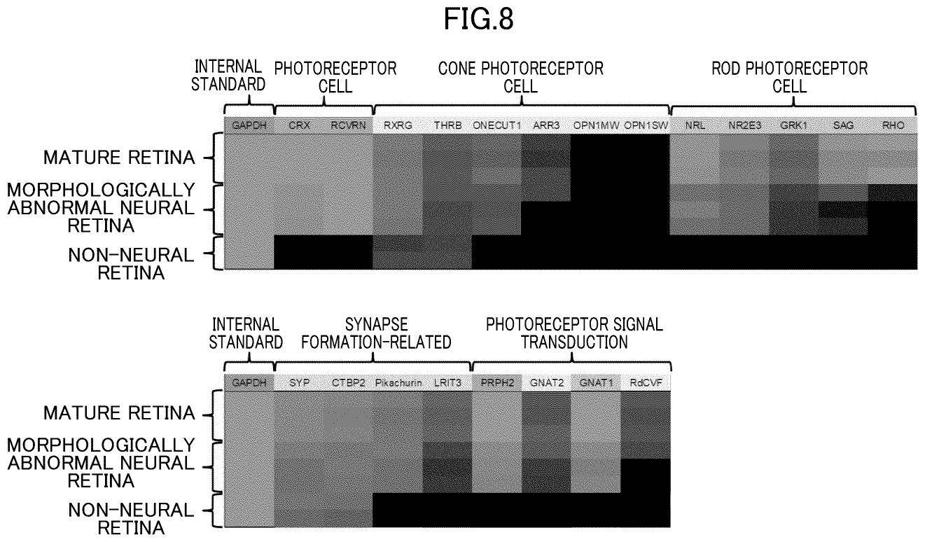

The results of the gene expression analysis are shown in FIGS. 8 (sheets) and 9 (rings) in a heat map data. The gene expression level was evaluated by the ΔCt value calculated from the difference between the Ct value of the target gene and the Ct value of GAPDH, which is the gene used as an internal standard.

ΔCt value=Ct value of target gene-Ct value of GAPDH

The lower the ΔCt value, the higher the gene expression level, and the higher the ΔCt value, the lower the gene expression level. Gray indicates a high gene expression level, and black indicates a low gene expression level (lighter colors indicate higher gene expression)
[1] Mature photoreceptor cell genes
[1-1] Cone photoreceptor cell genes: RXRG, THRB, ONECUT1, ARR3, OPNIMW (M-opsin), and OPN1SW (S-opsin)
[1-2] Rod photoreceptor cell genes NRL, NR2E3, GRK1, SAG, and Rhodopsin
[2] Synapse formation-related genes SYP, CTBP2, LRIT3 and
[3] Photoreceptor signal transduction genes: PRPH2, GNAT1, GNAT2, and RdCVF

When the gene expression of the mature neural retinal sheet was examined (FIG. 8), in addition to the photoreceptor cell markers Crx and Recoverin, the expression of mature photoreceptor cell genes, particularly rod photoreceptor cell genes NRL, NR2E3, GRK1, SAG, and Rhodopsin was observed. In addition, high expression of synapse formation-related genes SYP, CTBP2, Pikachurin, and LRIT3 was also observed. In addition, expression of photoreceptor signal transduction genes PRPH2, GNAT1, and RdCVF was also observed. From the above, the mature neural retinal sheet showed gene expression characteristic of photoreceptor cell maturation, and it was shown that the neural retinal sheet has photoreceptor cell maturation ability.

On the other hand, in the mature morphologically abnormal neural retinal sheet, expression of photoreceptor cell markers Crx and Recoverin was observed, but expression of mature photoreceptor cell genes, particularly rod photoreceptor cell genes GRK1, SAG, and Rhodopsin was not observed, and expression of NRL and NR2E3 was observed, but the expression tended to be lower than that of the mature neural retinal sheet. Similarly, although expression of synapse formation-related genes SYP, CTBP2, and LRIT3 and photoreceptor signal transduction genes PRPH2, GNAT1, and RdCVF was observed, the expression tended to be lower than that of the mature neural retinal sheet. In addition, the mature non-neural retinal sheet did not show expression of photoreceptor cell markers Crx and Recoverin, and did not show expression of mature photoreceptor cell genes, synapse formation-related genes, and photoreceptor signal transduction genes.

From these results, it was found that the photoreceptor cell maturation ability of the sheets can be compared by this test method.

A difference of 2 in the ΔCt value corresponds to a 4-fold difference in gene expression level. When the gene expression level after culture (after mature culture) increased by 4-fold or more compared to the gene expression level before culture in medium D (before mature culture) for a certain gene, maturation was considered to be confirmed for that gene. It was confirmed that a similar tendency was observed in the difference analysis.

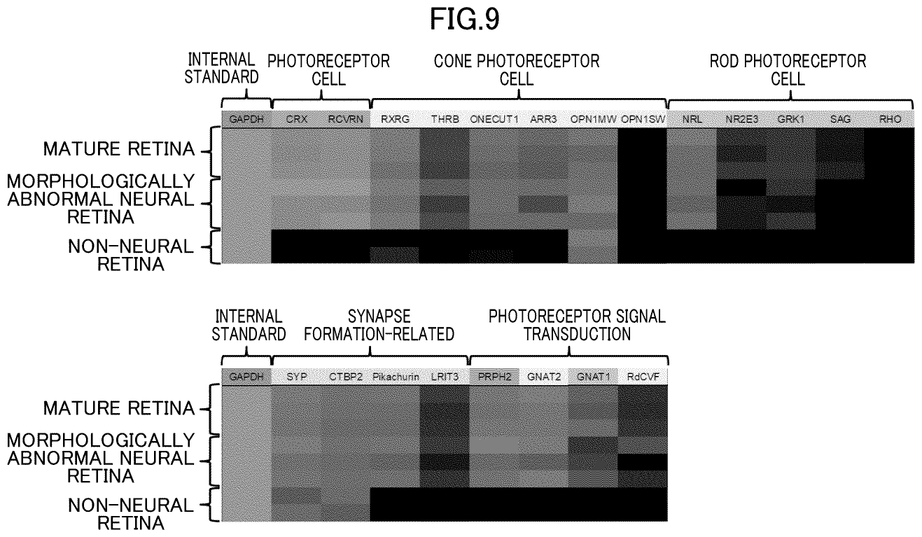

The gene expression level of the sheet after culture (after mature culture) was compared with the gene expression level of the ring before culture in medium D (before mature culture) (FIGS. 8 and 9). Regarding Rhodopsin, which is a mature photoreceptor cell gene marker, LRIT3, which is a synapse formation-related gene, and PRPH2, which is a photoreceptor signal transduction gene, the gene expression level was increased by 4-fold or more in the neural retina, whereas it was not increased by 4-fold or more in the morphologically abnormal neural retina and the non-neural retina. Therefore, photoreceptor cell maturation was not confirmed for these genes. It was confirmed that a similar tendency was observed in the difference analysis. From these results, it was demonstrated that the photoreceptor cell maturation ability evaluation method characterized by long-term culture newly developed this time is useful for evaluating the photoreceptor cell maturation ability of a neural retinal sheet.

### Test Example 6: Relationship Between Photoreceptor Cell Differentiation Ability and Photoreceptor Cell Maturation Ability

Neural retina was differentiated from human iPS cells by the method described in Test Example 1, and a total of 25 neural retinal sheet samples (#1 to #25) were prepared by the method described in Test Example 3. The prepared neural retinal sheets were stored in a commercially available storage solution for 4 days as described in Test Example 3.

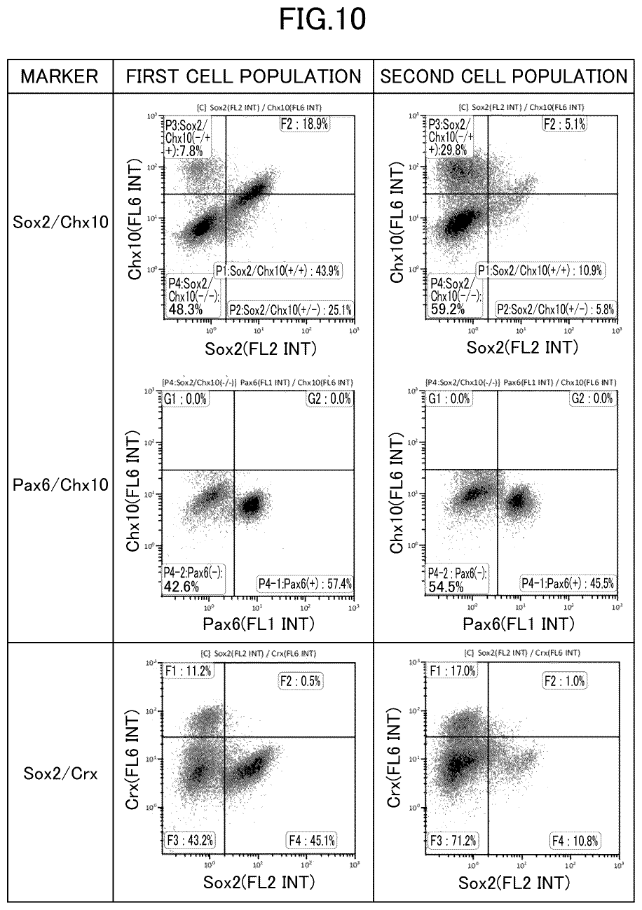

Of the obtained neural retinal sheets, “#1 to #11” were used as the first test samples, and neural retina “#12 to #21” were used as the second test samples, and cells were dispersed, fixed, stained, measured, and analyzed by the method described in Test Example 3 after culturing in the presence and absence of DAPT. The obtained measurement results are as shown in FIG. 10, and the calculation results of various neural retinal cells are shown in Table 7.

TABLE 7
Results of neural retinal sheet

| | Cell population | | | Fold |
|---|---|---|---|---|
| Cell type | 1 | 2 | Difference | change |
| Neural retinal progenitor | 40.2% | 7.1% | −33.1% | 0.18 |
| cells Sox2+/Chx10+ | | | | |
| Bipolar precursor cells | 7.8% | 29.8% | +22.0% | 3.82 |
| Sox2−/Chx10+ | | | | |
| Ganglion cells and amacrine | 24.5% | 23.9% | −0.6% | 0.98 |
| cells Pax6+/Sox2−/Chx10+ | | | | |
| Photoreceptor precursors and | 11.6% | 17.9% | +6.3% | 1.54 |
| photoreceptor cells Crx+ | | | | |
| Total | 84.1% | 78.7% | | |

Similar to the results in Test Example 4, in cell population 1 (control group) containing the neural retinal sheet, the proportion of neural retinal progenitor cells was 10% or more and photoreceptor precursors and photoreceptor cells were 5% or more even under the condition of no Notch signal inhibitor, so it is considered that the neural retinal sheet used in this test was an appropriate neural retina to be used as a test sample. In addition, neural retinal progenitor cells decreased from 40.2% to 7.1%, and photoreceptor precursors and photoreceptor cells increased from 11.6% to 17.9% by DAPT (Notch signal inhibitor) treatment. Thus, compared to cell population 1 (DAPT non-added group), in cell population 2 (DAPT added group), neural retinal progenitor cells decreased by 5% or more and photoreceptor precursors and photoreceptor cells increased by 5% or more in difference analysis, so it was determined that the neural retinal sheet had photoreceptor cell differentiation ability. In addition, in fold change, neural retinal progenitor cells decreased to 0.3-fold or less (decreased by 70% or more) and photoreceptor precursors and photoreceptor cells increased by 1.2-fold or more (increased by 20% or more), so it was determined that the neural retinal sheet had photoreceptor cell differentiation ability.

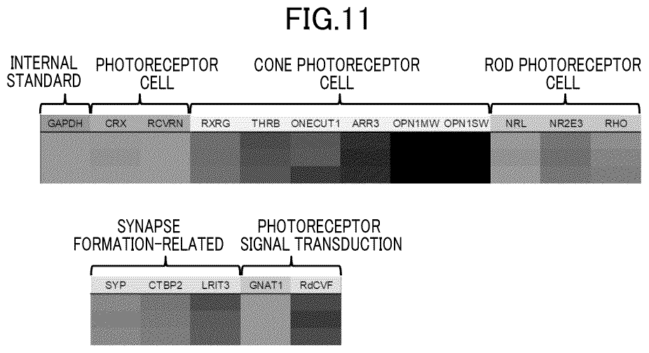

Next, the photoreceptor cell maturation ability of the lot of neural retinal sheets in which photoreceptor cell differentiation ability was confirmed was evaluated. Specifically, using neural retinal sheets #22 to #24, mature neural retinal sheets were obtained by the method described in Test Example 5, after which total RNA was extracted, followed by reverse transcription, a Pre-Run, and real-time PCR. The obtained measurement results are shown in FIG. 11.

When the gene expression of the mature neural retinal sheet was examined, in addition to the photoreceptor cell markers Crx and Recoverin, the expression of mature photoreceptor cell genes, particularly rod photoreceptor cell genes NRL, NR2E3, and Rhodopsin, was observed. In addition, high expression of synapse formation-related genes SYP, CTBP2, and LRIT3 was also observed. In addition, expression of photoreceptor signal transduction genes PRPH2, GNAT1, and RdCVF was also observed. The gene expression of Rhodopsin, which is a mature photoreceptor cell gene marker, and LRIT3, which is a synapse formation-related gene, was 4-fold or more compared to the gene expression level before the start of culture (before the start of mature culture). From the above, the mature neural retinal sheet showed gene expression characteristic of mature photoreceptor cells, and it was shown that the neural retinal sheet has photoreceptor cell maturation ability.

From these results, it was demonstrated that the lot of neural retinal sheets having photoreceptor cell differentiation ability also has photoreceptor cell maturation ability.

### Test Example 7: Verification of Photoreceptor Cell Maturation In Vivo of Neural Retinal Sheets Having Photoreceptor Cell Differentiation Ability and Photoreceptor Cell Maturation Ability

It was evaluated whether the neural retinal sheet, in which photoreceptor cell differentiation ability and photoreceptor cell maturation ability were observed in Test Example 6, actually engrafts and matures when administered in vivo.

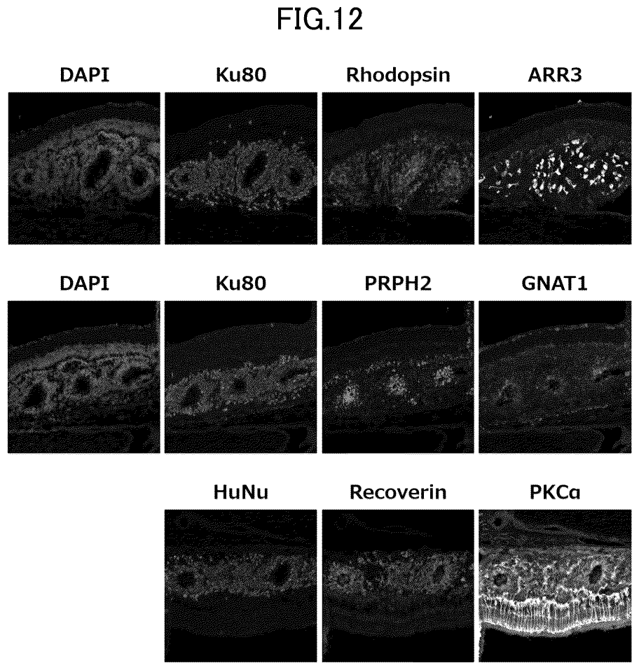

Of the neural retinal sheets prepared in Test Example 6, “#25” was washed three times with HBSS and then transplanted subretinally into retinal degeneration model rats (RCS rats, obtained from RRRC). After housing for 4 months, the eyeball containing the transplanted neural retinal sheet was removed. The removed eyeball was fixed with 4% paraformaldehyde (PFA fixation) and subjected to sucrose substitution. The fixed eyeball was used to prepare frozen sections using a cryostat. These frozen sections were immunostained for DAPI for staining nuclei, Ku80 (anti-Ku80 antibody, R&D Systems, goat), which is one of the markers for human cell nuclei, Rhodopsin (anti-RET-P1 antibody, Sigma-Aldrich, mouse), which is one of the markers for rod photoreceptor cells, ARR3 (anti-cone arrestin antibody, Novus Biologicals, goat), which is one of the markers for cone photoreceptor cells, PRPH2 (anti-PRPH2 antibody, Millipore, mouse), which is one of the markers for mature photoreceptor cell outer segments, Gnat1 (anti-Gnat1 antibody, Novus Biologicals, rabbit), which is one of the phototransduction markers, HuNu (anti-HuNu antibody, Millipore, mouse), which is one of the markers for human cell nuclei, Recoverin (anti-Recoverin antibody, Proteintech, rabbit), which is one of the markers for photoreceptor cells, and PKCα (anti-PKCα antibody, R&D Systems, goat), which is one of the markers for bipolar cells. The stained sections were observed using a fluorescence microscope (manufactured by Keyence Corporation), and immunostained images were acquired (FIG. 12).

From the results of FIG. 12, the transplanted and engrafted neural retinal sheet (graft) expressed Rhodopsin, which is a rod photoreceptor cell marker, and ARR3, which is a cone photoreceptor cell marker. That is, it was confirmed that the transplanted neural retinal sheet was normally differentiated and matured into rod photoreceptor cells and cone photoreceptor cells in vivo. Furthermore, the graft expressed PRPH2, which is a mature photoreceptor cell outer segment marker, and Gnat1, which is a phototransduction marker. That is, it was confirmed that the transplanted neural retinal sheet was normally matured into photoreceptor cells by forming outer segments in vivo. From these results, the neural retinal sheet determined as having photoreceptor cell differentiation ability and photoreceptor cell maturation ability by the method of the present invention engrafts under the retina of retinal degeneration model rats, and differentiates and matures into rod photoreceptor cells and cone photoreceptor cells after transplantation, expresses mature photoreceptor cell markers, and expresses photoreceptor-related genes, that is, it was demonstrated that it can differentiate and mature to the stage necessary for exerting visual function in the in vivo environment.

### Test Example 8: Evaluation of Photoreceptor Cell Differentiation Ability of Mature Natural Retinal Sheet

By evaluating the neural retina after mature culture, it was evaluated by the method described in Test Example 3 that the neural retinal sheet loses photoreceptor cell differentiation ability with maturation.

Neural retina was differentiated from human iPS cells by the method described in Test Example 1, and cultured at 37 °C under 5% CO₂ conditions until approximately day 150 after the start of suspension culture using medium B described in Test Example 1 or medium D described in Test Example 5, and 7 mature neural retinal sheet samples were prepared by the method described in Test Example 3. After storing the neural retinal sheet in a solution prepared by adding Gentacin (antibiotic) to a commercially available storage solution for 4 days, 3 samples were used as the first sample, and 4 samples were used as the second sample, and cultured in medium B and medium C described in Test Example 3 for 11 days, respectively, and dispersed, fixed, stained, measured, and analyzed. The analysis was performed by analysis method [2-1] and analysis method [3]. The calculated results are shown in Table 8.

TABLE 8
Results of mature neural retinal sheet

| | Cell population | | | Fold |
|---|---|---|---|---|
| Cell type | 1 | 2 | Difference | change |
| Neural retinal progenitor | 4.7% | 1.1% | −3.6% | 0.2 |
| cells Pax6+/Chx10+ | | | | |
| Bipolar precursor cells | 25.0% | 39.1% | +14.1% | 1.6 |
| Pax6−/Chx10+ | | | | |
| Ganglion cells and amacrine | 4.9% | 4.5% | −0.4% | 0.9 |
| cells Pax6+/Chx10− | | | | |
| Photoreceptor precursors and | 34.4% | 29.9% | −4.5% | 0.9 |
| photoreceptor cells Crx+ | | | | |
| Total | 69.0% | 74.6% | | |

As shown in Table 8, in cell population 1 (control group), the proportion of neural retinal progenitor cells was lower, and the proportion of bipolar precursor cells and photoreceptor precursors and photoreceptor cells was higher than that of cell population 1 in Tables 3 and 4. From this, it was confirmed that the differentiation of the neural retinal sheet was progressing. Neural retinal progenitor cells slightly decreased by DAPT (Notch signal inhibitor) treatment, but photoreceptor precursors and photoreceptor cells did not increase, so photoreceptor cell differentiation ability was not confirmed. More specifically, according to difference analysis, in cell population 2 (DAPT added group), neural retinal progenitor cells did not decrease by 5% or more and photoreceptor precursors and photoreceptor cells did not increase by 5% or more compared to cell population 1 (control group). In addition, according to fold change, in cell population 2 (DAPT added group), neural retinal progenitor cells decreased to 0.3-fold or less (decreased by 70% or more), but photoreceptor precursors and photoreceptor cells did not increase by 1.2-fold or more (increased by 20% or more) compared to cell population 1 (control group). Therefore, photoreceptor cell differentiation ability was not observed in the mature neural retinal sheet, and it was demonstrated that the presence or absence of photoreceptor cell differentiation ability can be distinguished by this evaluation method.

### Test Example 9: Comparative Verification of Photoreceptor Cell Maturation Ability Evaluation Method and Photoreceptor Cell Maturation In Vivo

Gene expression was compared to confirm that photoreceptor cell maturation of neural retinal sheets in vitro shown in Test Example 5 mimics maturation of neural retinal sheets in vivo.

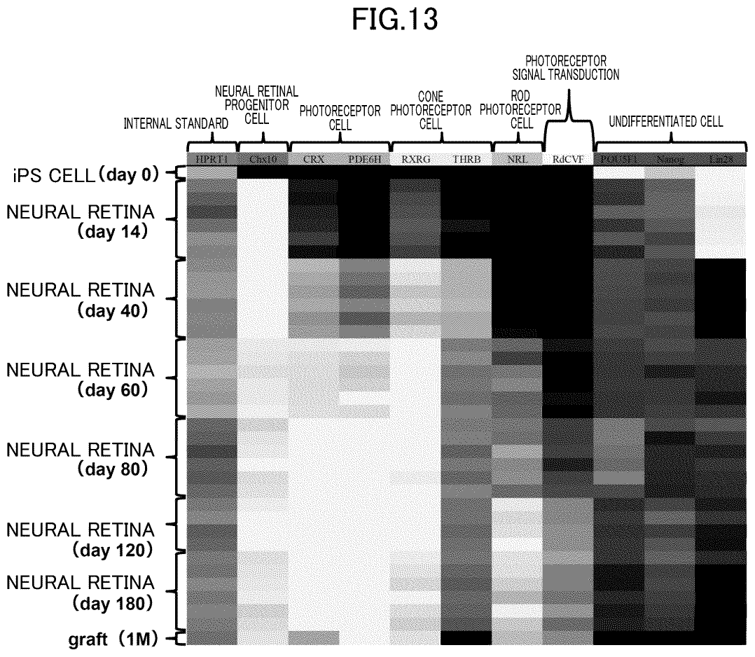

Neural retina was differentiated from human iPS cells by the method described in Test Example 1, and at the timing of approximately 14, 40, 60, and 80 days after the start of suspension culture, 6 neural retinal sheet samples were prepared by the method described in Example 3 from the neural retina in culture. The neural retinal sheet obtained on approximately day 80 after the start of suspension culture was stored in a solution prepared by adding Gentacin (antibiotic) to a commercially available storage solution for 3 to 4 days. Then, 10 samples were used for evaluation in vitro and 1 sample was used for evaluation in vivo. The 10 samples for evaluation in vitro were cultured until approximately day 120 or 180 after the start of suspension culture by the method described in Test Example 6 to obtain 4 and 6 mature neural retinal sheet samples, respectively. The sample for evaluation in vivo was transplanted subretinally into immunodeficient rats (nude rats, obtained from CLEA Japan, Inc.). After housing for about 1 month, the eyeball containing the transplanted neural retinal sheet was removed, and the transplantation site was dissected so that the rat eyeball portion was as small as possible. Total RNA was extracted from human iPS cells, mature neural retinal sheets after each culture day, and the eyeball transplantation site containing the neural retinal sheet, and reverse transcription, Pre-Run, and real-time PCR were performed. For gene expression analysis, only gene markers that do not react with mRNA derived from nude rats were used. Therefore, the Ct value of HPRT1 was used instead of the Ct value of GAPDH for the internal standard of gene expression analysis, and PDE6H was used instead of Recoverin for the photoreceptor cell marker. The probes used are shown in Table 9. The results are shown in FIG. 13 in a heat map data. Furthermore, additional evaluations were performed for MKi67, which is a proliferating cell marker, STX1A, which is an amacrine cell marker, and SOX9 and HES1, which are Müller glial cell markers. The PCR probes used for the additional measurement are shown in Table 9.

TABLE 9
Measured genes and their PCR probes

| Category | Gene | Probe ID | GenBank ID |
|---|---|---|---|
| Photoreceptor cell | PDH6H | Hs01124155_m1 | NM_006205.2 |
| | | | XM_017019431.1 |
| Proliferating cell | MKi67 | Hs04260396_g1 | NM_001145966.1 |
| | | | NM_002417.4 |
| | | | XM_006717864.3 |
| | | | XM_011539818.2 |
| Amacrine cell | STX1A | Hs00270282_m1 | NM_001165903.1 |
| | | | NM_004603.3 |
| | | | XM_017012567.1 |
| Müller glial cell | SOX9 | Hs00165814_m1 | NM_000346.3 |
| | HES1 | Hs00172878_m1 | NM_005524.3 |

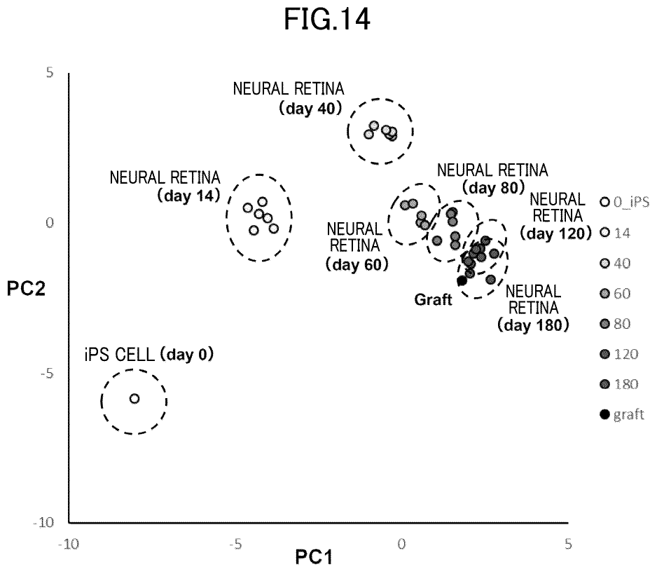

Principal component analysis (PCA) was performed on the value obtained by multiplying the obtained gene expression analysis result (ΔCt value) by −1. The result of PCA is shown in FIG. 14.

In iPS cells, expression of undifferentiated markers POU5F1, Nanog, and Lin28 was observed, and expression of other retinal markers was not confirmed. On the other hand, an increase in the expression of various retinal markers was observed as the number of differentiation days in suspension culture progressed. Chx10, which is a neural retinal progenitor cell marker, was observed on day 14 of differentiation, and gene expression of Crx and PDE6H, which are photoreceptor cell markers, was confirmed on and after day 40 of differentiation, and it was confirmed that the expression was sustained on subsequent differentiation days. Expression of mature photoreceptor cell genes, particularly rod photoreceptor cell gene NRL, and photoreceptor signal transduction gene RdCVF was observed on and after day 120 after differentiation. On the other hand, expression of mature photoreceptor cell genes, particularly rod photoreceptor cell gene NRL, and photoreceptor signal transduction gene RdCVF was also observed in the transplanted neural retinal sheet.

In addition, as a result of PCA, the first principal component (PC1) had a high positive contribution rate of genes related to photoreceptor cell maturation such as Crx, PDE6H, NRL, RXRG, and RdCVF, and a high negative contribution rate of genes related to undifferentiated state and proliferation such as Nanog, Lin28, and Mki67. In addition, the second principal component (PC2) had a high positive contribution rate of retinal cell genes other than photoreceptor cells such as Chx10, HES1, and STX1A. Therefore, PC1 moved in the positive direction as the number of differentiation days of the retinal tissue progressed, and PC2 moved in the positive direction as the number of differentiation days progressed, reached a maximum on day 40 of differentiation, and then moved in the negative direction. In addition, the transplantation site (graft) containing the transplanted retinal sheet and the mature retinal sheet on and after day 120 of differentiation were plotted at close positions, and it was found that there was similarity in gene expression. Therefore, it was found that photoreceptor cell maturation of neural retinal sheets in vitro mimics maturation of neural retinal sheets in vivo.

From the above, by culturing at least a part of a cell population containing a retinal graft in the presence of a Notch signal inhibitor and measuring a proportion of cells expressing photoreceptor cell markers including Crx and/or Recoverin, it was found that the differentiation ability of the retinal graft in vivo can be evaluated. In addition, by culturing at least a part of a cell population containing a retinal graft in the presence of retinoic acid and measuring the expression levels of (i) a photoreceptor cell maturation-related gene, (ii) a synapse formation-related gene, and (iii) a photoreceptor auxiliary gene, it was found that the maturation ability of the retinal graft in vivo can be evaluated. Therefore, it becomes possible to evaluate the quality of a retinal graft without actually transplanting it into the retina.

1. A method for evaluating quality of a retinal graft, comprising:
(1) preparing at least a part of a cell population containing the retinal graft as a test sample;
(2) culturing the test sample in the presence of a Notch signal inhibitor to obtain a test cell population;
(3) measuring a proportion of cells expressing photoreceptor cell markers including Crx and/or Recoverin in the test cell population; and
(4) comparing the proportion measured in step 3 with a proportion of cells expressing the photoreceptor cell markers measured in a control cell population.

2. The method according to claim 1, further comprising:
(3′) measuring a proportion of cells expressing additional cell markers including Chx10, Pax6, and/or Sox2 in the test cell population; and
(4′) comparing the proportion measured in the step 3′ with a proportion of cells expressing the additional cell markers measured in a control cell population.

3. The method according to claim 1, wherein the Notch signal inhibitor includes at least one selected from the group consisting of DAPT, crenigacestat, LY411575, dibenzazepine, RO4929097, semagacestat, and IMR-1A.

4. The method according to claim 1, wherein in step 2, the test sample is cultured for 2 days to 21 days.

5. The method according to claim 1, wherein
the cell population containing the retinal graft is
derived from pluripotent stem cells, and/or
a cell population obtained by culturing pluripotent stem cells by an SFEB method or an SFEBq method for 50 to 90 days, and/or
a spherical cell aggregate or a sheet-like cell aggregate.

6. The method according to claim 1, wherein in step 3, the proportion of the cells is measured by flow cytometry.

7. The method according to claim 2, wherein in step 3′, the proportion of the cells is measured by flow cytometry.

8. The method according to claim 1, for identifying a retinal graft containing retinal progenitor cells having an ability to differentiate into photoreceptor cells in vivo and/or an ability to mature in vivo.

9. The method according to claim 1, wherein a ratio of the number of Crx-positive cells to the total number of cells in the test sample in step 1 is 5% or more and 15% or less.

10. The method according to claim 1, wherein the retinal graft is determined as containing retinal progenitor cells having an ability to differentiate into photoreceptor cells in vivo and/or an ability to mature in vivo in any of the following cases:
when the proportion of cells expressing the photoreceptor cell marker in the test cell population is increased by 5% or more compared to the control cell population by difference analysis, or
when the proportion of cells expressing the photoreceptor cell marker in the test cell population is increased by 1.2-fold or more compared to the control cell population by fold change analysis.

11. The method according to claim 2, wherein the retinal graft is determined as containing retinal progenitor cells having an ability to differentiate into photoreceptor cells in vivo and/or an ability to mature in vivo in any of the following cases:
when the proportion of cells expressing the photoreceptor cell marker in the test cell population is increased by 5% or more compared to the control cell population, and the proportion of cells expressing the additional cell marker in the test cell population is decreased by 5% or more compared to the control cell population by difference analysis, or
when the proportion of cells expressing the photoreceptor cell marker in the test cell population is increased by 1.2-fold or more compared to the control cell population, and the proportion of cells expressing the additional cell marker in the test cell population is decreased to 0.3-fold or less compared to the control cell population by fold change analysis.

12. A method for evaluating quality of a retinal graft, comprising:
(A) preparing at least a part of a cell population containing the retinal graft as a test sample;
(B) culturing the test sample in the presence of retinoic acid to obtain a test cell population;
(C) measuring expression levels of (i) a photoreceptor cell maturation-related gene, (ii) a synapse formation-related gene, and (iii) a photoreceptor auxiliary gene in the test cell population; and
(D) comparing the expression levels of the photoreceptor cell maturation-related gene, the synapse formation-related gene, and the photoreceptor auxiliary gene measured in step C with expression levels in the test sample before the culture of step B,
wherein the photoreceptor cell maturation-related gene includes a cone photoreceptor cell maturation-related gene including at least one selected from the group consisting of RXRG, THRB, ONECUT1, ARR3, OPN1MW (M-opsin), and OPN1SW (S-opsin), and/or a rod photoreceptor cell maturation-related gene including at least one selected from the group consisting of NRL, NR2E3, GRK1, SAG, and RHO;
the synapse formation-related gene includes at least one selected from the group consisting of SYP, CTBP2, Pikachurin, and LRIT3; and
the photoreceptor auxiliary gene includes at least one selected from the group consisting of PRPH2, GNAT1, GNAT2, and RdCVF.

13. The method according to claim 12, wherein in step B, the test sample is cultured for 30 days to 150 days.

14. The method according to claim 12, wherein the photoreceptor cell maturation-related gene includes a cone photoreceptor cell maturation-related gene including at least one selected from the group consisting of RXRG, THRB, ONECUT1, ARR3, OPNIMW (M-opsin), and OPN1SW (S-opsin), and/or a rod photoreceptor cell maturation-related gene including at least one selected from the group consisting of NRL, NR2E3, GRK1, SAG, and RHO.

15. The method according to claim 12, wherein the synapse formation-related gene includes SYP, CTBP2, Pikachurin, and LRIT3, and/or
the photoreceptor auxiliary gene includes PRPH2, GNAT1, GNAT2, and RdCVF.

16. The method according to claim 12, for identifying a retinal graft containing retinal progenitor cells having the ability to mature into rod photoreceptor cells or cone photoreceptor cells in vivo.

17. A method for producing a retinal graft, comprising:
(I) obtaining a cell population containing a retinal graft from pluripotent stem cells;
(II) evaluating quality of the retinal graft contained in the cell population obtained in step (I) by the method according to claim 1; and
(III) determining whether or not the retinal graft contained in the cell population is of a quality suitable for transplantation based on the evaluation result of step (II).
